(19) United States
(12) Patent Application Publication
(10) Pub. No.: US 2017/0362590 A1
(43) Pub. Date: Dec. 21, 2017

(54) PHARMACEUTICAL COMPOSITIONS COMPRISING MICRORNA

(71) Applicants: Sudarshan Anand, Portland, OR (US); Cristina Espinosa-Diez, Portland, OR (US)

(72) Inventors: Sudarshan Anand, Portland, OR (US); Cristina Espinosa-Diez, Portland, OR (US)

(73) Assignee: OREGON HEALTH & SCIENCE UNIVERSITY, PORTLAND, OR (US)

(21) Appl. No.: 15/442,251

(22) Filed: Feb. 24, 2017

Related U.S. Application Data
(60) Provisional application No. 62/300,615, filed on Feb. 26, 2016.

(51) Int. Cl. C12N 15/113; A61K 31/713

(52) U.S. Cl. CPC C12N 15/113; A61K 31/713; C12N 2310/141; C12N 2310/14

(57) ABSTRACT

Pharmaceutical compositions comprising one or more of miR-494-3p, miR-99b-5p, and/or miR-21-3p and a pharmaceutically acceptable carrier as well as methods of using said compositions to treat cancer characterized by aberrant activity of an MRN complex.

### PRIORITY CLAIM

This application claims the benefit of U.S. Provisional Patent Application 62/300,615, filed 26 Feb. 2016 and entitled PHARMACEUTICAL COMPOSITIONS COMPRISING MICRORNA. This application is incorporated by reference herein in its entirety.

### ACKNOWLEDGEMENT OF GOVERNMENT SUPPORT

This invention was made with the support of the United States government under the terms of grant number 4R00HL112962-03 awarded by the National Institutes of Health. The United States government has certain rights to this invention.

### FIELD

Generally, the field is pharmaceutical compositions comprising polynucleotides. More specifically, the field is pharmaceutical compositions comprising miRNA that silence components of an MRN complex.

### BACKGROUND

Preclinical and clinical studies have revealed that tumor endothelium is abnormal, resistant to genotoxic stress, and as such, functions as a key determinant of therapeutic responses to radiation and chemotherapy. While it is well established that radiation and chemotherapy cause DNA damage in tumor vasculature, the molecular mechanisms leading to subsequent cell cycle arrest, apoptosis or senescence in vascular cells are poorly understood. Therefore, identifying and understanding factor(s) that mediate DNA damage responses in tumor endothelial cells can provide potential targets for sensitizing tumor vasculature to radiation and other DNA damaging agents and improve their therapeutic efficacy in cancer.

### SUMMARY

Disclosed herein are pharmaceutical compositions comprising an effective amount of a polynucleotide with a sequence of miR-494-3p, miR-99b-5p, and/or miR-21-3p and a pharmaceutically acceptable carrier. In some embodiments, the miR-494-3p, miR-99b-5p, and/or miR-21-3p are provided in a recombinant expression vector. The recombinant expression vector can be any appropriate vector such as a plasmid or viral vector. Exemplary viral vectors include adenoviral, adeno associated viral, lentiviral, herpesviral, or poxviral vectors.

In other embodiments, the polynucleotide comprises at least one modified nucleotide. In still other embodiments, the pharmaceutical composition comprises polyribonucleotides of miR-494-3p and miR-99b-5p, miR-494-3p and miR-21-3p, miR-99b-5p and miR-21-3p, or all of miR-494-3p, miR-99b-5p, and miR-21-3p.

Such pharmaceutical compositions can be produced for use in the treatment of a cancer characterized by aberrant MRN complex activity.

Also disclosed are methods of treating a cancer characterized by aberrant MRN complex activity in a subject. Such a method involves administering an effective amount of the disclosed pharmaceutical compositions to the subject.

Also disclosed are methods of inhibiting angiogenesis activity in a subject. Such a method involves administering an effective amount of the disclosed pharmaceutical compositions to the subject.

### BRIEF DESCRIPTION OF THE SEVERAL VIEWS OF THE DRAWINGS

FIGS. 1A-1D collectively show HUVEC cells transfected with the indicated miR mimics or negative control miRNA (NC). For all of 1A-1D, bars represent means±SEM of 3 biological replicates. * indicates P<0.05.

FIG. 1A is a graph showing results of senescence measured 48h post-transfection using a senescence associated β-galactosidase activity assay. Bars represent % blue positive cells in at least 100 cells per experiment (n≧10

FIG. 1B is an image of a representative western blot showing phospho-p21 and pRb levels in HUVEC cell lysates after 48 hours transfection.

FIG. 1C is an image (top) and graph (bottom) showing sprouting angiogenesis measured at 6d post transfection in a 3D bead assay and visualized using FITC-lectin staining. The area was quantified across at least 20 beads—quantification results are shown in the graph. * indicates P<0.05

FIG. 1D is a graph showing % telomerase activity measured using a kit as described herein. Results were normalized with protein concentration. Bars depict means±SEM of 3 biological replicates. * indicates P<0.05.

For FIGS. 2A-2G, * indicates P<0.05.

FIG. 2A is a table of expression of the indicated mRNAs in the presence of the indicated miRs. HUVECs were transfected as described in FIG. 1A above. RNA isolated at 24h post transfection was reverse transcribed and evaluated on a TaqMan Gene signature qRTPCR-Panel. The top mRNAs commonly downregulated with all three miRs are shown.

FIG. 2B is a graph of the results of a MirTrap™ assay depicting enrichment of miR-494-3p target mRNAs in 293T cells

FIG. 2C is a graph showing the results of a luciferase assays depicting a decrease in luciferase-3′UTR expression in 293T cells 24h post co-transfection of plasmid with the human MRE11a-3′-UTR and indicated miR mimics. Bars depict mean±Bars depict mean miR tivity of 3 independent biological replicates.

FIG. 2D is a graph showing the results of a luciferase assays depicting a decrease in luciferase-3′UTR expression in 293T cells 24h post co-transfection of 125 ng of pMIR plasmid with the human RAD50-3′-UTR and indicated miR mimics (40 nM). Bars depict mean±Bars depict mean miR mimics the results of a luciferase assays depic * indicates P<0.05

FIG. 2E is a graph showing the results of a luciferase assays depicting a decrease in luciferase-3′UTR expression in 293T cells 24h post co-transfection of 125 ng of pMIR plasmid with the human NBN-3′-UTR and indicated miR mimics (40 nM). Bars depict mean±Bars depict mean miR mimics the results of a luciferase assays depic * indicates P<0.05

FIG. 2F is a graph showing the mRNA levels at 24h where HUVECs were transfected as indicated below. Bar graph represents mean±ean of at least 3 biological replicates. mRNA levels are normalized to housekeeping control GAPDH. * indicates P<0.05.

FIG. 2G is an image of a representative western blot showing protein levels at where HUVECs were transfected for 48 hours as indicated and cell lysates were collected with RIPA lysis buffer.

For FIGS. 3A-3D, HUVECs were transfected with siRNAs against the indicated genes. * indicates P<0.05.

FIG. 3A is a bar graph showing senescence in HUVECs transfected after 48 hours post-transfection with the indicated siRNAs and their corresponding siRNA control. Bars depict % positive cells in at least 100 cells per experiment, for n≧100* indicates P<0.05.

FIG. 3B is a bar graph of % telomerase activity in HUVECs transfected with the indicated siRNAs. Results were normalized with protein concentration. Bars depict means±SEM of 3 biological replicates. * indicates P<0.05.

FIG. 3C is an image of a representative western blot showing expression of the indicated proteins in HUVECs transfected for 48h with the indicated siRNAs.

FIG. 3D is an image and a graph of the results of angiogenic sprouting in a 3D bead assay. Images show representative beads for each condition. Bar graphs depict ImageJ quantitation of sprouting angiogenesis total area in more than 25 beads per experiment and condition (mean±ean). * indicates P<0.05.

FIG. 3E is a bar graph showing the results where HUVECs were transfected with a miR-494-3p mimic with or without an MRE11a specific target protector that prevents miR-494 mediated degradation of MRE11a. Senescence was assessed as indicated before at 48h post transfection.

FIG. 4A is an image of a representative western blot showing expression of the indicated proteins in cells transfected with the indicated microRNA inhibitors compared with a control inhibitor. Cells lysates were collected 48 h after transfection and assayed for MRE11a and RAD50 expression.

FIG. 4B is a graph showing expression of the indicated mRNAs in cells transfected with the indicated miR inhibitors. Bar graph represents mRNA levels normalized to housekeeping control GAPDH.

FIG. 4C is a plot showing the results where HUVECs were maintained for the indicated number of passages and expression of the indicated miRs measured. microRNA expression was measured by qPCR with specific Taqman assays for every microRNA. Results are plotted as fold change normalized with the housekeeping gene U6.

FIG. 4D is a plot showing the mRNA levels of the indicated genes results in HUVECs maintained for the indicated number of passages mRNA levels were normalized to housekeeping control GAPDH

FIG. 4E is a bar graph showing P18 HUVECs transfected with the indicated miR inhibitors and assessed for senescent phenotype 48h later. Bars represent % blue positive cells in at least 100 cells per experiment (n≧10. * indicates P<0.05.

FIG. 4F is a bar graph showing P18 HUVECs transfected with the indicated miR inhibitors and assessed for proliferation using a luminescence assay (Cell titer glo).

FIG. 4G is a bar graph showing P18 HUVECs transfected with the indicated miR inhibitors and assessed for cell death via caspase 3/7 activity using a luminescence assay (Caspase 3/7 Glo).

FIG. 5A is an image and plot of microRNA In situ hybridization for miR-494-3p in a human breast cancer tissue array comparing tumor area and adjacent normal area from the same patient. miR-494 expression was quantified with Image) software and normalized to the number of nuclei.

FIG. 5B is a plot of an analysis of the impact of MRN expression on overall survival from the human breast cancer TCGA dataset. A majority of the alterations in the MRN altered group were gain of copy number or gene expression increase.

FIG. 5C is a graph of 4T1 tumor weights on day 21 after orthotopic mammary fat pad implantation of either control or miR-494-3p transfected tumors. N=8 mice per group and * indicates P<0.05.

FIG. 6 is a bar graph representing miR-494 plasma levels after 5 h post-radiation (2 gy dose) in HCT116 xenograft tumor model in athymic nude mice. microRNA expression (mean±ean) was measured by qPCR with specific Taqman assays. Results are plotted normalized with the housekeeping gene U6. * indicates P<0.05.

FIG. 7A is a plot showing pri-miR-494 (squares) and pri-miR-99b (circles) expression in HUVECs 1 and 3 h post 2 Gy radiation as measured by qPCR.

FIG. 7B is a plot of a pri-miRNA expression radiation-dose response. Graph shows pri-miR-99b and pri-miR-494 at 1 h post-radiation exposition to 2, 5, 10 and 20 Gy.

FIG. 7C is a graph showing the results of a β-Gal assay in HUVEC transfected for 48 hours with miR-99b and miR-494. Graph shows the mean percentage+SEM of β-gal positive cells. At least 100 cells were counted per assay.

FIG. 7D is a graph showing the results of a telomerase activity assay. HUVEC were transfected with miR-99b and miR-494 during 48 hours. Bars depict the mean percentage+SEM of Telomerase activity.

FIG. 7E is an image showing a representative western-blot of p21 and pRB in HUVEC.

FIG. 7F is an image and graph depicting the results of a fibrin bead 3D assay. Transiently transfected HUVEC were cultured with fibrin-beads in presence of Smooth Muscle Cells over 5 days. The images show representative beads for each condition. Bars depict mean±SEM of lectin area analyzed across at least 25 beads per group.

FIG. 7G is an image showing CD31 staining in vivo. Matrigel plugs were implated in nude mice and treated with miR-NC or miR-494 7C1 nanoparticles after 4 days, for 2 consecutive days. Mices were sacrificed at day 7 and plugs were harvested for tissue sections. Angiogenesis was measured by staining matrigel plugs sections with anti-CD31 (green) and aSMA (red) and DAPI (blue). Quantification of CD31 area from at least three mice per group are shown. Bars show mean±SEM. *p≦0.05.

FIG. 8A shows the results of miR-TRAP immunoprecipitation. Bars depict mean of mRNA levels+SEM analyzed by qRT-PCR in HEK-293T transfected with miR-99b and miR-494 for 24 hours.

FIG. 8B shows the results of target validation in HUVEC. Graphs depict mean mRNA levels+SEM of the three MRN complex members.

FIG. 8C shows a representative western-blot of MRE11a and RAD50 after 48 hours microRNA transfection.

FIG. 8D shows luminescence from 3-UTR-luciferase constructs for MRE11a, RAD50 and NBS 24 h after transfection with miR-99b or miR-494. Graph represents mean+SEM of 3 independent experiments.

FIG. 8E shows MRN complex expression in vivo. Matrigel plugs were implated in nude mice and treated with miR-NC or miR-494 7C1 nanoparticles after 4 days, for 2 consecutive days. Mice were sacrificed at day 6 and plugs were harvested for RNA isolation. Graph represents mean+SEM of mRNA levels of Mre11a, Rad50 and Nbs. *p≦0.05.

FIG. 9A shows the results of a β-Gal assay in HUVEC transfected for 48 hours with different siRNAs against MRE11a, RAD50 and NBN and the respective siRNA control. Graph shows % mean+SEM of β-gal positive cells for at least hundred cells analyzed.

FIG. 9B shows the results of a telomerase activity assay. HUVEC were transfected with a target protector for miR-494 binding site in HUVEC transfected for 48 hours with specific siRNAs against MRE11a, RAD50 and NBN and the respective siRNA control. Bars depict % mean+SEM of Telomerase activity.

FIG. 9C shows the results of a Fibrin bead 3D assay. The images show representative beads for each condition. Bars depict mean+SEM of E-lectin area analyzed across at least 25 beads per group.

FIG. 9D shows the results of a β-Gal assay in HUVEC treated with Mirin 1 for 48 hours.

Graph shows % mean+SEM of β-gal positive cells for at least hundred cells analyzed.

FIG. 9E shows the results of a cell proliferation assay in HUVECs treated with VEGFR2 inhibitor Vandetanib (10 μM) alone or in combination with Mirin-1 (50 μM).

FIG. 10A shows the results of a β-Gal assay in HUVEC co-transfected for 48 hours with miR-99b or miR-494 and VEGFR2 receptor gapmer.

FIG. 10B shows the results of a β-Gal assay in HUVEC co-transfected for 48 hours with siRNAs against MRE11a or NBN and VEGFR2 receptor gapmer. Graph shows % mean+SEM of β-gal positive cells for at least one hundred cells analyzed.

FIG. 10C shows the results of a cell proliferation assay in HUVECs transfected for 48 hours with VEGFR2 gapmer and treated with Mirin1 (50 μM) for 24 hours.

FIG. 10D shows the results of a β-Gal assay in HUVEC transfected for 48 hours with VEGFR2 gapmer and treated with Mirin1 (50 μM) for 24 hours. Graphs show % mean±SEM of β-gal positive cells for more than 100 hundred cells analyzed. *p≦0.05.

FIG. 10E shows the results of a cell proliferation assay in HUVECs treated with VEGFR2 inhibitor Vandetanib (10 μM) alone or in combination with Mirin-1 (50 μM).

FIG. 11A shows the mRNA levels of VEGF-early response genes after miR-99b expression. HUVECs were transfected for a total of 48 hours. At 24 hours cells were starved overnight and then treated 3 hours with VEGF (50 ng/ml). Graphs depict mean mRNA levels+SEM.

FIG. 11B shows a representative western blot of miR-99b VEGF-dependent signaling. The western blot shows P-ERK, total ERK and β-actin. All HUVECs were starved overnight before VEGF treatment. Bar graphs represent densitometric analysis of P-ERK/ERK/Actin.

FIG. 11C shows a representative western blot of HUVEC treated with MRN complex inhibitor Mirin-1 in VEGF-dependent signaling. All HUVECs were starved overnight before VEGF treatment. Bar graphs represent densitometric analysis of P-ERK/ERK/Actin.

FIG. 12 shows the senescence phenotype in cells treated with conditioned media. A β-Gal assay in HUVEC and ASMC cultured for 48 hours with conditioned media from miR-99b and miR-494 transfected HUVECs. Graphs show % mean±SEM of β-gal positive cells for at least hundred cells analyzed.

FIG. 13A is a heat map representing the 3 common mRNAs down-regulated by miR-99b and miR-494 analyzed by TaqMan q-PCR with a Human DNA Repair Array.

FIG. 13B depicts microRNA inhibition in HUVECs affect MRN levels. HUVEC were transfected with miR-99b or miR-494 inhibitor during 24 hours.

FIG. 13C is a representative western blot for MRE11a and RAD50 after 48 hours transfection.

FIG. 14A shows the results of a β-Gal assay in HUVEC transfected with a target protector for miR-494 binding site in MRE11a-3′-UTR or an scramble control, and the corresponding miRNAs for 48 hours. Graph shows % mean+SEM of β-gal positive cells for at least hundred cells analyzed.

FIG. 14B shows the results of a telomerase activity assay. HUVEC were transfected with a target protector for miR-494 binding site in MRE11a-3′-UTR or an scramble control, and the corresponding miRNAs for 48 hours. Bars depict % mean+SEM of telomerase activity.

FIG. 14C shows the results of HUVEC transfected with a target protector for miR-494 binding site in MRE11a-3′-UTR or an scramble control, and the corresponding miRNAs during 24 hours. Bar graph depict mean+SEM of the three members of MRN complex.

FIG. 14D shows the results where HUVEC were transfected with specific siRNAs against MRE11a, RAD50 and NBN and the respective siRNA control during 24 hours. Bar graph depict mean+SEM of the three members of MRN complex.

FIG. 14E shows a representative western blot of MRE11a, RAD50 and P-Rb in HUVEC transfected with specific siRNAs against MRE11a, RAD50 and NBN and the respective siRNA control during 48 hours.

FIG. 15A is a heat map represents the 7 mRNAs commonly changed by miR-99b, miR-494, siRNA-MRE11a and siRNA-RAD50 analyzed by TaqMan q-PCR with a human senescence gene array.

FIG. 15B is a representative CD44 histogramam analyzed by flow cytometry. HUVECs were transfected for 48 hours with miR-494 and its respective control.

FIG. 16A shows Pecam1 and miR-494 expression in vivo. Matrigel plugs were implated in nude mice and treated with miR-NC or miR-494 7C1 nanoparticles after 4 days, for 2 consecutive days. Mices were sacrificed at day 6 and plugs were harvested for RNA isolation. Graph represents mean+SEM of mRNA levels of Pecam1 and miR-494.

FIG. 16B is a representative image of matrigel plugs stained with Mre11a (red). Matrigel plugs were treated with miR-NC and miR-494 7C1 conjugated nanoparticles nanoparticles after 4 days, for 2 consecutive days. Mices were sacrificed at day 6 and plugs were harvested for frozen sections and stained with Mre11a antibody.

FIG. 17A shows mRNA levels of MRN complex expression in HUVECs in the indicated number of passages.

FIG. 17B shows levels of miR-99b and miR-494 expression in HUVECs in different passages.

FIG. 17C is a graph with bars depicting the % mean±SEM of β-gal positive cells of at least hundred cells analyzed. HUVEC in passage 25 were transfected with microRNA inhibitors for 48 hours.

FIG. 17D is a graph showing the cell titer of HUVEC (P25) transfected with microRNA inhibitors for 72 h. *p≦0.05.

FIG. 17E is a graph showing the results of a caspase 7 assay performed in HUVEC (P25) transfected with microRNA inhibitors for 72 h. *p≦0.05.

FIG. 18A is a plot of the Time response of miR-99 and miR-494 expression after 1 and 6 hours treatment with VEGF. HUVECs were starved overnight and then treated with VEGF (50 ng/ml).

FIG. 18B is an image of a western blot showing the Phosphorylation of ATM, H2AX and p38 MAP kinase 10 min after treatment of starved HUVECs.

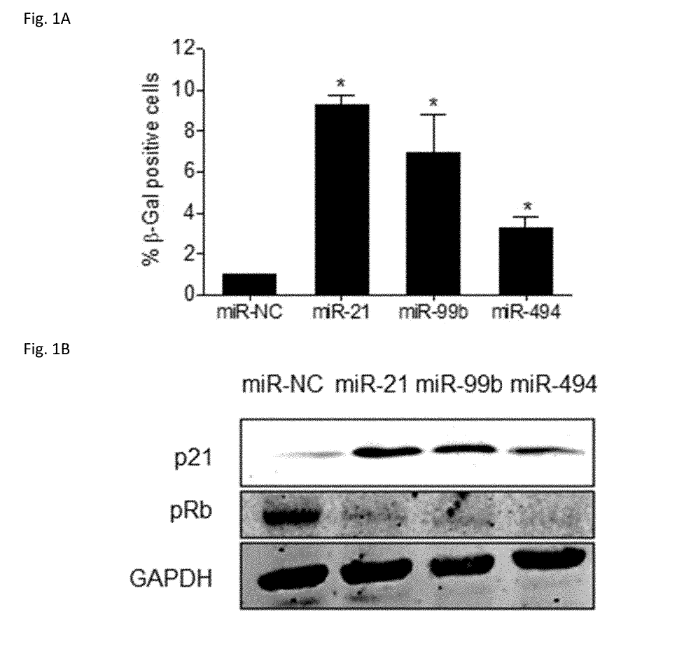

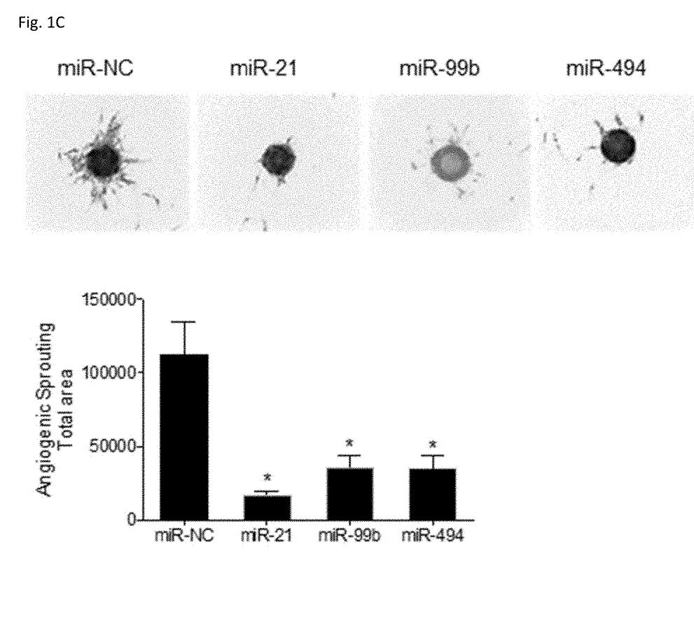

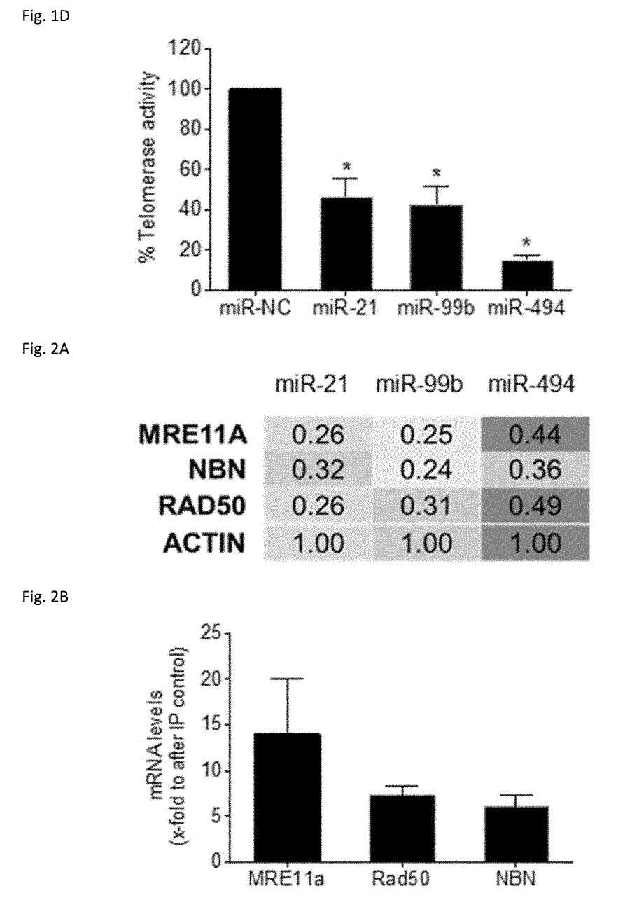

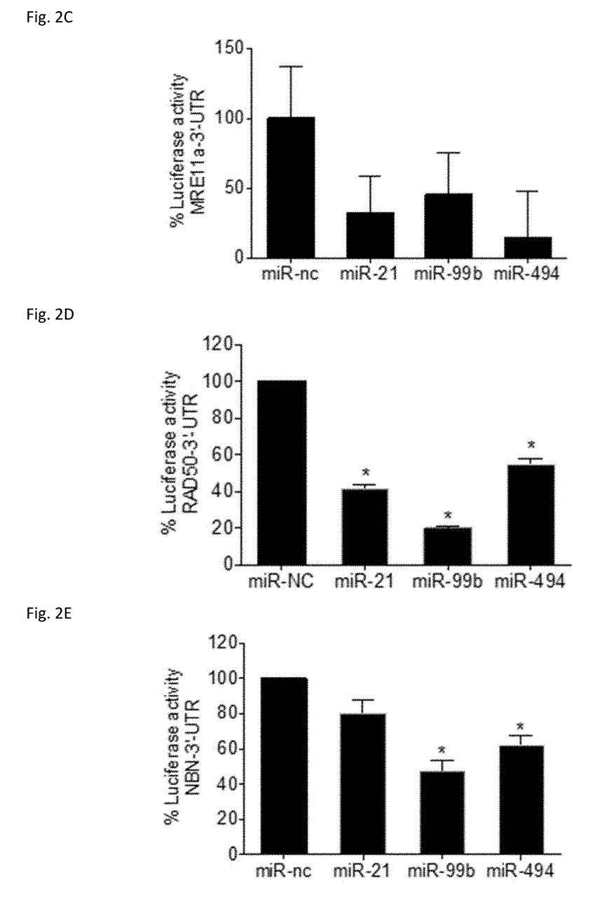

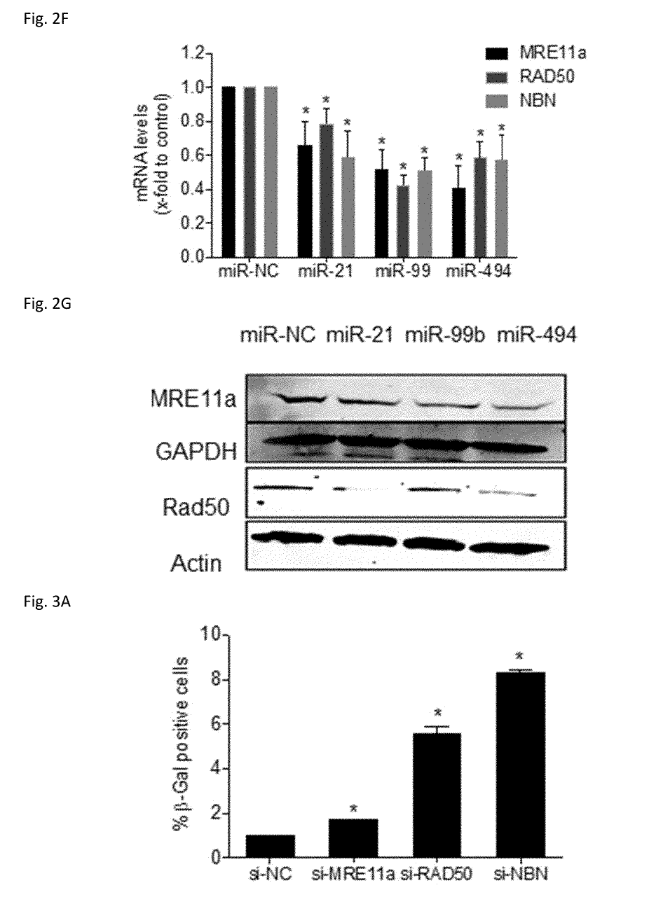

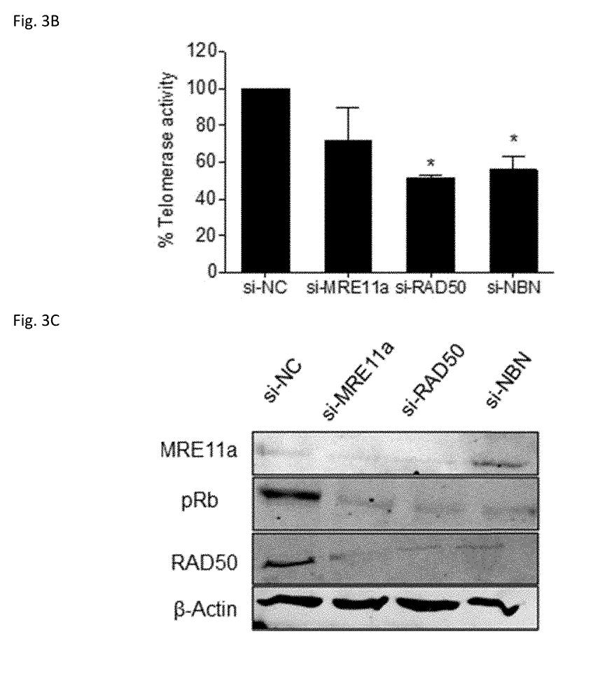

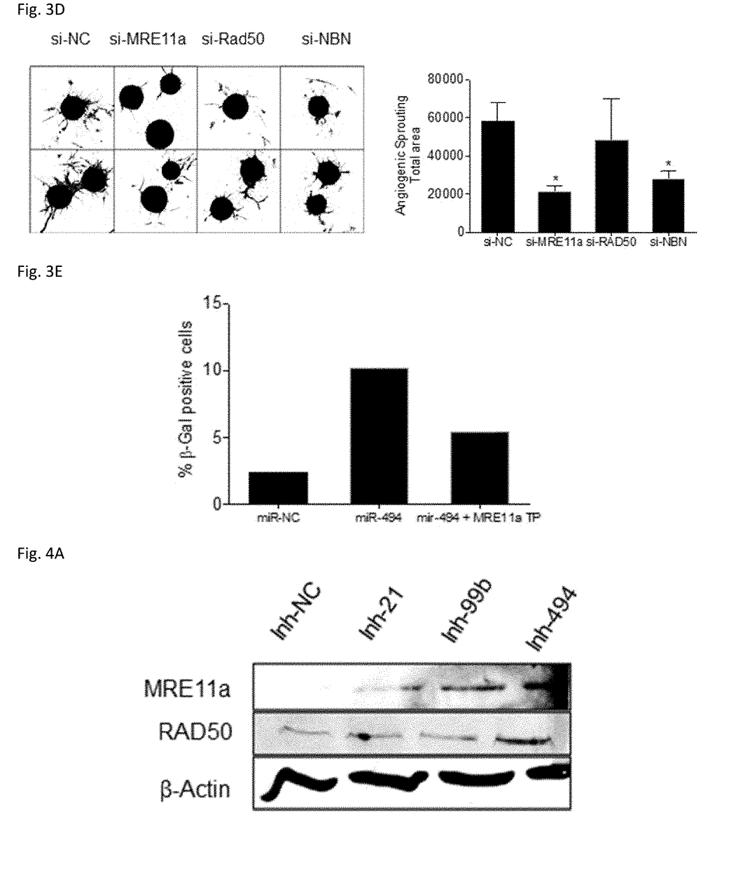

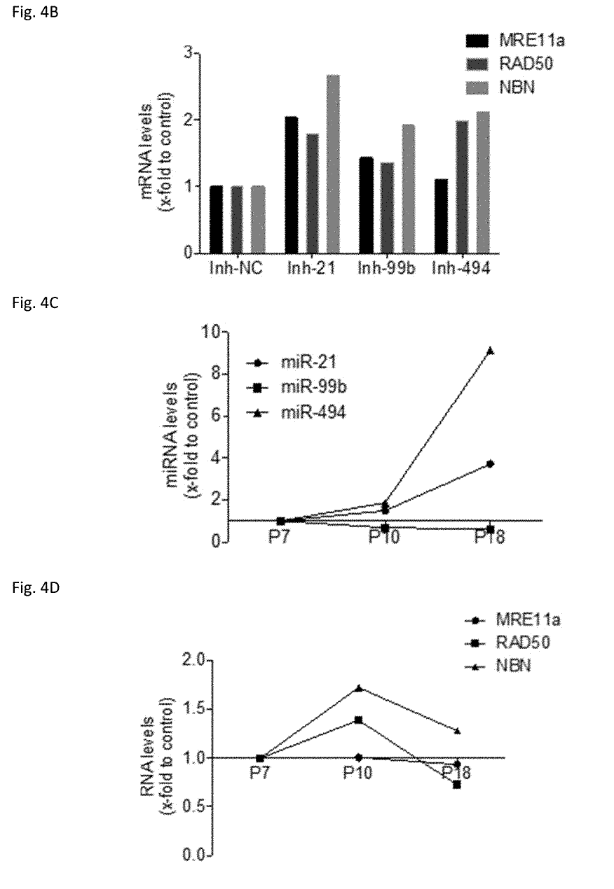

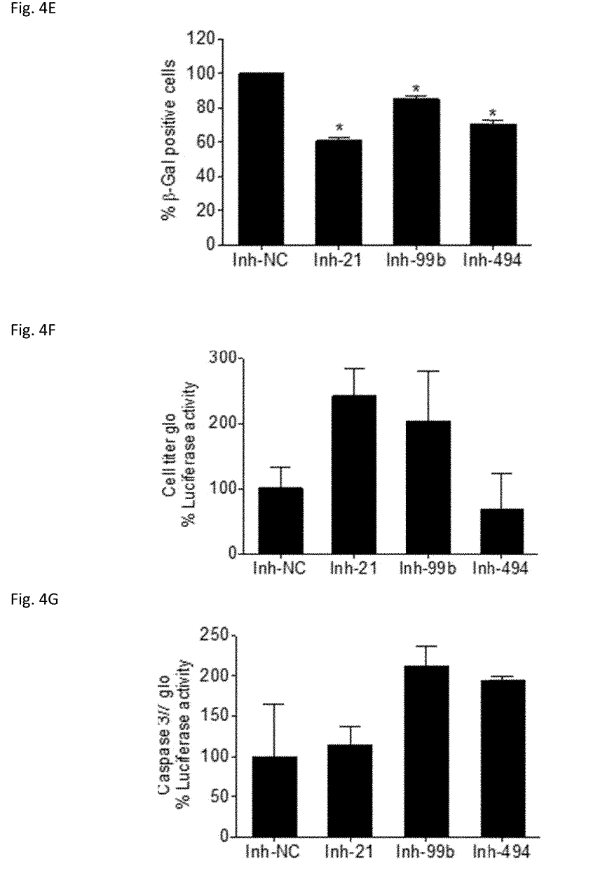

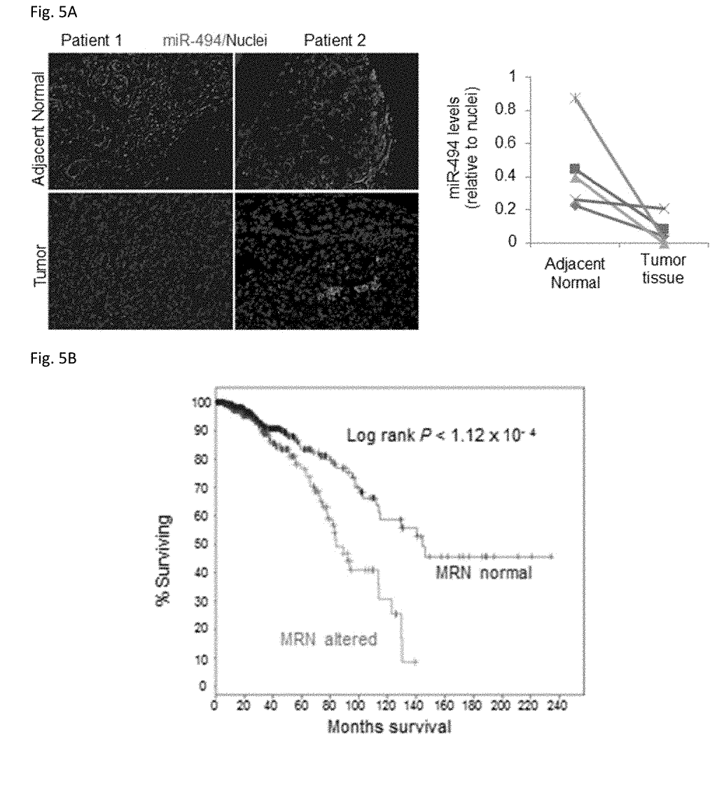

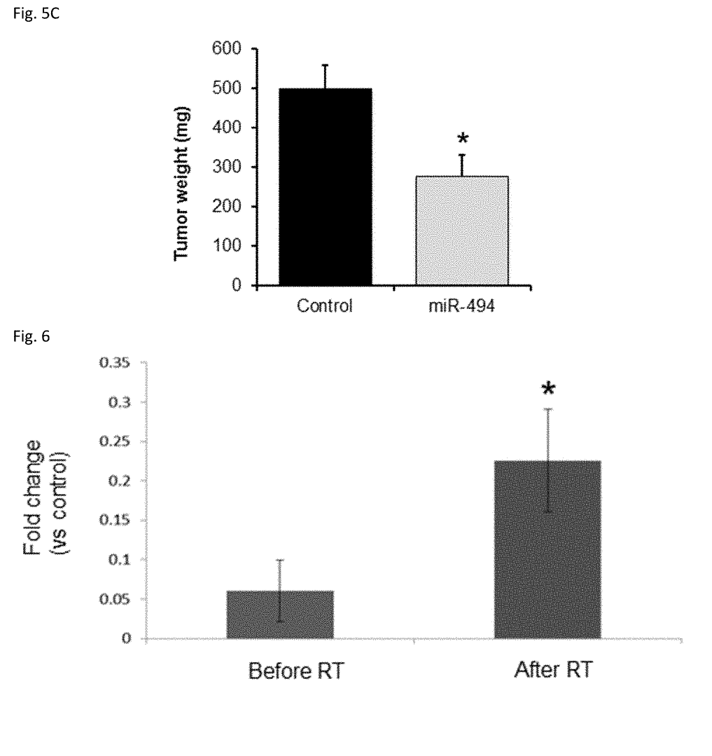

### SEQUENCE LISTING

SEQ ID NO: 1 is the sequence of Human miR-494-3p.

SEQ ID NO: 2 is the sequence of Human miR-21-3p.

SEQ ID NO: 3 is the sequence of Human miR-99b-5pb-5p.

### DETAILED DESCRIPTION

### Definitions

Unless otherwise noted, technical terms are used according to conventional usage.

Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” In addition, the materials, methods, and examples are illustrative only and not intended to be limiting. In order to facilitate review of the various embodiments of the disclosure, the following explanations of specific terms are provided:

Administration: To provide or give a subject an agent, such as a composition comprising an effective amount of a pharmaceutical composition comprising miR-494-3p, miR-21-3p, and/or miR-99b-5p. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Control: A reference standard. As disclosed herein, a control can be a negative control such as a microRNA mimic. Such a mimic can be an oligoribonucleotide not known to silence any known gene and particularly genes silenced by miR-494-3p, miR-21-3p, and/or miR-99b-5p.

Effective amount: As used herein, the term “effective amount” refers to an amount of an agent, such as a pharmaceutical composition comprising miR-494-3p, miR-21-3p, and/or miR-99b-5p that is sufficient to generate a desired response, such as reduce or eliminate a sign or symptom of a condition or disease such as a reduction of aberrant MRN complex activity or any downstream results of aberrant MRN complex activity in a cancer characterized by such activity. In some examples, an “effective amount” is one that treats (including prophylaxis) one or more symptoms and/or underlying causes of any of a disorder or disease. An effective amount can be a therapeutically effective amount, including an amount that prevents one or more signs or symptoms of a particular disease or condition from developing, such as one or more signs or symptoms associated with cancer, particularly cancer characterized by aberrant MRN complex activity.

Expression: The process by which the coded information of a gene is converted into an operational, non-operational, or structural part of a cell, such as the synthesis of RNA such as a microRNA or messenger RNA from the gene.

MicroRNA: MicroRNAs are a major class of biomolecules involved in control of gene expression. For example, in human heart, liver or brain, miRNAs play a role in tissue specification or cell lineage decisions. In addition, miRNAs influence a variety of processes, including early development, cell proliferation and cell death, and apoptosis and fat metabolism. The large number of miRNA genes, the diverse expression patterns and the abundance of potential miRNA targets suggest that miRNAs may be a significant source of genetic diversity.

A mature miRNA is typically an 18-25 nucleotide non-coding RNA that regulates expression of an mRNA including sequences complementary to the miRNA. These small RNA molecules are known to control gene expression by regulating the stability and/or translation of mRNAs. For example, miRNAs bind to the 3′ UTR of target mRNAs and suppress translation. MiRNAs may also bind to target mRNAs and mediate gene silencing through the RNAi pathway. MiRNAs may also regulate gene expression by causing chromatin condensation.

A miRNA silences translation of one or more specific mRNA molecules by binding to a miRNA recognition element (MRE,) which is defined as any sequence that directly base pairs with and interacts with the miRNA somewhere on the mRNA transcript. Often, the MRE is present in the 3′ untranslated region (UTR) of the mRNA, but it may also be present in the coding sequence or in the 5′ UTR. MREs are not necessarily perfect complements to miRNAs, usually having only a few bases of complementarity to the miRNA and often containing one or more mismatches within those bases of complementarity. The MRE may be any sequence capable of being bound by a miRNA sufficiently that the translation of a gene to which the MRE is operably linked (such as a CMV gene that is essential or augmenting for growth in vivo is repressed by a miRNA silencing mechanism such as the RISC. An microRNA can interchangeably be abbreviated to ‘miRNA’ or ‘miR’.

Nucleic acid molecules: A deoxyribonucleotide or ribonucleotide polymer including, without limitation, cDNA, mRNA, genomic DNA, and synthetic (such as chemically synthesized) DNA. The nucleic acid molecule can be double-stranded or single-stranded. Where single-stranded, the nucleic acid molecule can be the sense strand or the antisense strand. In addition, nucleic acid molecule can be circular or linear.

Nucleotide sequences or nucleic acid sequences: The terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid can be single-stranded, or partially or completely double stranded (duplex). Duplex nucleic acids can be homoduplex or heteroduplex.

Operably Linked: A first nucleic acid sequence is operably linked with a second nucleic acid sequence when the first nucleic acid sequence is placed in such a way that it has an effect upon the second nucleic acid sequence. For instance, a MRE is operably linked to a coding sequence that it silences if binding of the miRNA to the MRE silences the expression of the coding sequence. Operably linked DNA sequences may be contiguous, or they may operate at a distance.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polynucleotide: As used herein, the term “polynucleotide” refers to a polymer of ribonucleic acid (RNA) or deoxyribonucleic acid (DNA). A polynucleotide is made up of four bases; adenine, cytosine, guanine, and thymine/uracil (uracil is used in RNA). A coding sequence from a nucleic acid is indicative of the sequence of the protein encoded by the nucleic acid. A polyribonucleotide refers to a polymer of ribonucleic acid.

Promoter: A promoter may be any of a number of nucleic acid control sequences that directs transcription of a nucleic acid. Typically, a eukaryotic promoter includes necessary nucleic acid sequences near the start site of transcription, such as, in the case of a polymerase II type promoter, a TATA element or any other specific DNA sequence that is recognized by one or more transcription factors. Expression by a promoter may be further modulated by enhancer or repressor elements. Numerous examples of promoters are available and well known to those of skill in the art. A nucleic acid comprising a promoter operably linked to a nucleic acid sequence that codes for a particular polypeptide can be termed an expression vector.

Recombinant: A recombinant nucleic acid or polypeptide is one that has a sequence that is not naturally occurring or has a sequence that is made by an artificial combination of two or more otherwise separated segments of sequence, for example a CMV vector comprising a heterologous antigen and/or made replication deficient by the addition of a miRNA response element operably linked to a CMV gene that is essential or augmenting for growth in vivo. This artificial combination is often accomplished by chemical synthesis or, more commonly, by the artificial manipulation of isolated segments of nucleic acids, e.g., by genetic engineering techniques. A recombinant polypeptide can also refer to a polypeptide that has been made using recombinant nucleic acids, including recombinant nucleic acids transferred to a host organism that is not the natural source of the polypeptide (for example, nucleic acids encoding polypeptides that form a CMV vector comprising a heterologous antigen).

Sample (or biological sample): A biological specimen containing microRNA that is obtained from a subject.

Subject: Living multi-cellular vertebrate organisms, a category that includes human and non-human mammals. These include animals used in research models mice, rats, and non-human primates such as monkeys, animals used in agriculture such as cattle, pigs, and other livestock, and companion animals such as dogs or cats. In some examples a subject is a human patient, including a patient with cancer.

Disclosed herein is a pharmaceutical composition comprising an effective amount one or more microRNAs of miR-494-3p, miR-99b-5p, and miR-21-3p and a pharmaceutically acceptable carrier. Also disclosed are methods of treating a cancer characterized by aberrant MRN complex activity comprising administering such a pharmaceutical composition.

Treatment: As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

miRNA

A microRNA silences translation of one or more specific mRNA molecules by binding to a microRNA recognition element (MRE,) which is defined as any sequence that directly base pairs with and interacts with the microRNA somewhere on the mRNA transcript. Often, the MRE is present in the 3′ untranslated region (UTR) of the mRNA, but it may also be present in the coding sequence or in the 5′ UTR. MREs are not necessarily perfect complements to microRNAs, usually having only a few bases of complementarity to the microRNA and often containing one or more mismatches within those bases of complementarity. As a result, microRNA-mRNA interactions are difficult to predict. The MRE may be any sequence capable of being bound by a microRNA sufficiently that the translation of the target mRNA is repressed by a microRNA silencing mechanism such as the RISC.

miRNA molecules can be provided in several forms including, e.g., as one or more isolated miRNA duplexes, as longer double-stranded RNA (dsRNA), or as miRNA or dsRNA transcribed from a transcriptional cassette in a DNA plasmid. The miRNA sequences may have overhangs (as 3′ or 5′ overhangs as described in Elbashir et al, Genes Dev 15, 188 (2001) or Nykänen et al, Cell 107, 309 (2001)) or may lack overhangs (i.e., have blunt ends).

One or more DNA plasmids encoding one or more miRNA templates may be used to provide miRNA. miRNA can be transcribed as sequences that automatically fold into duplexes with hairpin loops from DNA templates in plasmids having RNA polymerase III transcriptional units, for example, based on the naturally occurring transcription units for small nuclear RNA U6 or human RNase P RNA H1 (Brummelkamp et al, Science 296, 550 (2002); Donzé et al, Nucleic Acids Res 30, e46 (2002); Paddison et al, Genes Dev 16, 948 (2002); Yu et al, Proc Natl Acad Sci USA 99, 6047 (2002); Lee et al, Nat Biotech, 20, 500 (2002); Miyagishi et al, Nat Biotech 20, 497 (2002); Paul et al, Nat Biotech, 20, 505 (2002); and Sui et al, Proc Natl Acad Sci USA, 99, 5515 (2002)). Typically, a transcriptional unit or cassette will contain an RNA transcript promoter sequence, such as an H1-RNA or a U6 promoter, operably linked to a template for transcription of a desired miRNA sequence and a termination sequence, comprised of 2-3 uridine residues and a polythymidine (T5) sequence (polyadenylation signal) (Brummelkamp et al (2002) supra). The selected promoter can provide for constitutive or inducible transcription. Compositions and methods for DNA-directed transcription of RNA interference molecules are described in detail in U.S. Pat. No. 6,573,099. The transcriptional unit is incorporated into a plasmid or DNA vector from which the interfering RNA is transcribed. Plasmids suitable for in vivo delivery of genetic material for therapeutic purposes are described in detail in U.S. Pat. Nos. 5,962,428 and 5,910,488. The selected plasmid can provide for transient or stable delivery of a nucleic acid to a target cell. It will be apparent to those of skill in the art that plasmids originally designed to express desired gene sequences can be modified to contain a transcriptional unit cassette for transcription of miRNA.

Methods for isolating RNA, synthesizing RNA, hybridizing nucleic acids, making and screening cDNA libraries, and performing PCR are well known in the art (see, e.g., Gubler and Hoffman, Gene 25, 263-269 (1983); Sambrook and Russell, Molecular Cloning: A Laboratory Manual, Cold Spring Harbor Laboratory Press, Cold Spring Harbor N.Y., (2001)) as are PCR methods (see, U.S. Pat. Nos. 4,683,195 and 4,683,202; PCR Protocols: A Guide to Methods and Applications, Innis et al, eds, (1990)). Expression libraries are also well known to those of skill in the art. Additional basic texts disclosing the general methods of use in this invention include Sambrook and Russell (2001) supra; Kriegler, Gene Transfer and Expression: A Laboratory Manual (1990); and Current Protocols in Molecular Biology (Ausubel et al., eds., 1994).

A miRNA molecule can be chemically synthesized. A single-stranded nucleic acid that includes an miRNA sequence can be synthesized using any of a variety of techniques known in the art, such as those described in Usman et al, J Am Chem Soc, 109, 7845 (1987); Scaringe et al, Nucl Acids Res, 18, 5433 (1990); Wincott et al, Nucl Acids Res, 23, 2677-2684 (1995); and Wincott et al, Methods Mol Bio 74, 59 (1997). Synthesis of the single-stranded nucleic acid makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 micromolar scale protocol with a 2.5 min coupling step for 2′-O-methylated nucleotides. Alternatively, syntheses at the 0.2 micromolar scale can be performed on a 96-well plate synthesizer from Protogene. However, a larger or smaller scale of synthesis is encompassed by the invention, including any method of synthesis now known or yet to be disclosed. Suitable reagents for synthesis of the miRNA single stranded molecules, methods for RNA deprotection, and methods for RNA purification are known to those of skill in the art.

A double stranded miRNA can also be synthesized via a tandem synthesis technique, wherein both strands are synthesized as a single continuous fragment or strand separated by a linker that is subsequently cleaved to provide separate fragments or strands that hybridize to form a duplex. The linker may be any linker, including a polynucleotide linker or a non-nucleotide linker. The tandem synthesis of miRNA can be readily adapted to both multiwell/multiplate synthesis platforms as well as large scale synthesis platform employing batch reactors, synthesis columns, and the like. Alternatively, the miRNA can be assembled from two distinct single-stranded molecules, wherein one strand includes the sense strand and the other includes the antisense strand of the miRNA. For example, each strand can be synthesized separately and joined together by hybridization or ligation following synthesis and/or deprotection. Either the sense or the antisense strand can contain additional nucleotides that are not complementary to one another and do not form a double stranded miRNA. In certain other instances, the miRNA molecules can be synthesized as a single continuous fragment, where the self-complementary sense and antisense regions hybridize to form a miRNA duplex having hairpin secondary structure.

An miRNA molecule may comprise a duplex having two complementary strands that form a double-stranded region with least one modified nucleotide in the double-stranded region. The modified nucleotide may be on one strand or both. If the modified nucleotide is present on both strands, it may be in the same or different positions on each strand. A modified miRNA may be less immunostimulatory than a corresponding unmodified miRNA sequence, but retains the capability of silencing the expression of a target sequence.

Examples of modified nucleotides suitable for use in the present invention include, but are not limited to, ribonucleotides having a 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, or 2′-C-allyl group. Modified nucleotides having a conformation such as those described in the art, for example in Saenger, Principles of Nucleic Acid Structure, Springer-Verlag Ed. (1984), are also suitable for use in miRNA molecules. Other modified nucleotides include, without limitation: locked nucleic acid (LNA) nucleotides, G-clamp nucleotides, or nucleotide base analogs. LNA nucleotides include but need not be limited to 2′-O, 4′-Cmethylene-(D-ribofuranosyl)nucleotides), 2′-O-(2-methoxyethyl) (MOE) nucleotides, 2′-methyl-thio-ethyl nucleotides, 2′-deoxy-2′-fluoro (2′F) nucleotides, 2′-deoxy-2′-chloro (2C1) nucleotides, and 2′-azido nucleotides. A G-clamp nucleotide refers to a modified cytosine analog wherein the modifications confer the ability to hydrogen bond both Watson-Crick and Hoogsteen faces of a complementary guanine nucleotide within a duplex (Lin et al, J Am Chem Soc, 120, 8531-8532 (1998)). Nucleotide base analogs include for example, C-phenyl, C-naphthyl, other aromatic derivatives, inosine, azole carboxamides, and nitroazole derivatives such as 3-nitropyrrole, 4-nitroindole, 5-nitroindole, and 6-nitroindole (Loakes, Nucl Acids Res, 29, 2437-2447 (2001)).

A miRNA molecule may comprise one or more chemical modifications such as terminal cap moieties, phosphate backbone modifications, and the like. Examples of classes of terminal cap moieties include, without limitation, inverted deoxy abasic residues, glyceryl modifications, 4′,5′-methylene nucleotides, 1-(β-D-erythrofuranosyl) nucleotides, 4′-thio nucleotides, carbocyclic nucleotides, 1,5-anhydrohexitol nucleotides, L-nucleotides, α-nucleotides, modified base nucleotides, threopentafuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3-aminopropyl phosphate, 6-aminohexyl phosphate, 1,2-aminododecyl phosphate, hydroxypropyl phosphate, 1,4-butanediol phosphate, 3′-phosphoramidate, 5′-phosphoramidate, hexylphosphate, aminohexyl phosphate, 3′-phosphate, 5′-amino, 3′-phosphorothioate, 5′-phosphorothioate, phosphorodithioate, and bridging or nonbridging methylphosphonate or 5′-mercapto moieties (see, e.g., U.S. Pat. No. 5,998,203; Beaucage et al, Tetrahedron 49, 1925 (1993)). Non-limiting examples of phosphate backbone modifications (i.e., resulting in modified internucleotide linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions (see, e.g., Hunziker et al, Modern Synthetic Methods, VCH, 331-417 (1995); Mesmaeker et al, Antisense Research, ACS, 24-39 (1994)). Such chemical modifications can occur at the 5′-end and/or 3′-end of the sense strand, antisense strand, or both strands of the miRNA.

The sense and/or antisense strand of a miRNA may comprise a 3′-terminal overhang having 1 to 4 or more 2′-deoxyribonucleotides and/or any combination of modified and unmodified nucleotides. Additional examples of modified nucleotides and types of chemical modifications that can be introduced into the modified miRNA molecules of the present invention are described, e.g., in UK Patent No. GB 2,397,818 B and U.S. Patent Publication Nos. 20040192626 and 20050282188.

A miRNA molecule may comprise one or more non-nucleotides in one or both strands of the miRNA. A non-nucleotide may be any subunit, functional group, or other molecular entity capable of being incorporated into a nucleic acid chain in the place of one or more nucleotide units that is not or does not comprise a commonly recognized nucleotide base such as adenosine, guanine, cytosine, uracil, or thymine, such as a sugar or phosphate.

Chemical modification of the miRNA may also comprise attaching a conjugate to the miRNA molecule. The conjugate can be attached at the 5′- and/or the 3′-end of the sense and/or the antisense strand of the miRNA via a covalent attachment such as a nucleic acid or non-nucleic acid linker. The conjugate can also be attached to the miRNA through a carbamate group or other linking group (see, e.g., U.S. Patent Publication Nos. 20050074771, 20050043219, and 20050158727). A conjugate may be added to the miRNA for any of a number of purposes. For example, the conjugate may be a molecular entity that facilitates the delivery of the miRNA into a cell or the conjugate a molecule that comprises a drug or label. Examples of conjugate molecules suitable for attachment to the miRNA of the present invention include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof (see, e.g., U.S. Patent Publication Nos. 20030130186, 20040110296, and 20040249178; U.S. Pat. No. 6,753,423). Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in U.S. Patent Publication Nos. 20050119470 and 20050107325. Other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules described in U.S. Patent Publication No. 20050153337. Additional examples of conjugate molecules include a hydrophobic group, a membrane active compound, a cell penetrating compound, a cell targeting signal, an interaction modifier, or a steric stabilizer as described in U.S. Patent Publication No. 20040167090. Further examples include the conjugate molecules described in U.S. Patent Publication No. 20050239739.

The type of conjugate used and the extent of conjugation to the miRNA can be evaluated for improved pharmacokinetic profiles, bioavailability, and/or stability of the miRNA while retaining activity. As such, one skilled in the art can screen miRNA molecules having various conjugates attached thereto to identify miRNA conjugates having improved properties using any of a variety of well-known in vitro cell culture or in vivo animal models.

A miRNA may be incorporated into a carrier systems containing the miRNA molecules described herein. The carrier system can be a lipid-based carrier system such as a stabilized nucleic acid-lipid particle 5 (e.g., SNALP or SPLP), cationic lipid or liposome nucleic acid complexes (i.e., lipoplexes), a liposome, a micelle, a virosome, or a mixture thereof. In other embodiments, the carrier system is a polymer-based carrier system such as a cationic polymer-nucleic acid complex (i.e., polyplex). In additional embodiments, the carrier system is a cyclodextrin-based carrier system such as a cyclodextrin polymer-nucleic acid complex (see US Patent Application Publication 20070218122). In further embodiments, the carrier system is a protein-based carrier system such as a cationic peptide-nucleic acid complex. A miRNA molecule can also be delivered as modified or unmodified naked miRNA.

### Pharmaceutical Compositions

The miRNA compounds disclosed herein may be included in pharmaceutical compositions (including therapeutic and prophylactic formulations), typically combined together with one or more pharmaceutically acceptable carriers (known equivalently as vehicles) and, optionally, other therapeutic ingredients.

Such pharmaceutical compositions can formulated for administration to subjects by a variety of mucosal administration modes, including by oral, rectal, intranasal, intrapulmonary, intravitrial, or transdermal delivery, or by topical delivery to other surfaces including the eye. Optionally, the compositions can be administered by non-mucosal routes, including by intramuscular, subcutaneous, intravenous, intra-arterial, intra-articular, intraperitoneal, intrathecal, intracerebroventricular, or parenteral routes. In other examples, the compound can be administered ex vivo by direct exposure to cells, tissues or organs originating from a subject.

To formulate the pharmaceutical compositions, the compound can be combined with various pharmaceutically acceptable additives. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween®-80), solubility enhancing agents (for example, cyclodextrins and derivatives thereof), stabilizers (for example, serum albumin), and reducing agents (for example, glutathione) can be included.

When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7. The compound can be dispersed in any pharmaceutically acceptable carrier, which can include a hydrophilic compound having a capacity to disperse the compound, and any desired additives. The carrier can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a carrier, for example, polylactic acid, poly(lactic acid-glycolic acid) copolymer, polyhydroxybutyric acid, poly(hydroxybutyric acidglycolic acid) copolymer and mixtures thereof.

Alternatively or additionally, synthetic fatty acid esters such as polyglycerin fatty acid esters, sucrose fatty acid esters and the like can be employed as carriers. Hydrophilic polymers and other vehicles can be used alone or in combination, and enhanced structural integrity can be imparted to the vehicle by partial crystallization, ionic bonding, cross-linking and the like. The carrier can be provided in a variety of forms, including fluid or viscous solutions, gels, pastes, powders, microspheres, and films for direct application to a mucosal surface.

The compound can be combined with the carrier according to a variety of methods, and release of the compound can be by diffusion, disintegration of the vehicle, or associated formation of water channels. In some circumstances, the compound is dispersed in microcapsules (microspheres) or nanoparticles prepared from a suitable polymer, for example, 5-isobutyl 2-cyanoacrylate (see, for example, Michael et al., J. Pharmacy Pharmacol. 43, 1-5, (1991), and dispersed in a biocompatible dispersing medium, which yields sustained delivery and biological activity over a protracted time.

Pharmaceutical compositions for administering the compound can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

In certain embodiments, the compound can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the compound and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acidco-glycolic acid), poly(D-lactic acid-co-glycolic acid), and poly(L-lactic acid-coglycolic acid). Other useful biodegradable or bioerodible polymers include, but are not limited to, such polymers as poly(epsilon-caprolactone), poly(epsilon-caprolactone-CO-lactic acid), poly(epsilon.-aprolactone-CO-glycolic acid), poly(betahydroxy butyric acid), poly(alkyl-2-cyanoacrylate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

### Treatment

Disclosed herein are methods of treating a subject with cancer through administration of a pharmaceutical composition comprising an effective amount of one or more of miR-494-3p, miR-99b-5p, and/or miR-21-3p or a modified version thereof. The composition can be administered through any appropriate route including orally, parenterally, or topically. Administration also includes buccal, sublingual, sublabial administration as well as administration by inhalation.

The administration of a pharmaceutical composition comprising the disclosed compounds can be for prophylactic or therapeutic purposes. For prophylactic and therapeutic purposes, the treatments can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the treatments for viral infection can be provided as repeated doses within a prolonged prophylaxis or treatment regimen that will yield clinically significant results to alleviate one or more symptoms or detectable conditions associated with cancer, particularly cancer characterized by aberrant activity of an MRN complex.

An effective amount or concentration of the disclosed compounds may be any amount of a composition that alone, or together with one or more additional therapeutic agents, is sufficient to achieve a desired effect in a subject. The effective amount of the agent will be dependent on several factors, including, but not limited to, the subject being treated and the manner of administration of the therapeutic composition. In one example, a therapeutically effective amount or concentration is one that is sufficient to prevent advancement, delay progression, or to cause regression of a disease, or which is capable of reducing symptoms caused by any disease, including cancers characterized by aberrant activity of an MRN complex.

The actual effective amount will vary according to factors such as the particular status of the subject (for example, the subject's age, size, fitness, extent of symptoms, susceptibility factors, and the like) time and route of administration, other drugs or treatments being administered concurrently, as well as the specific pharmacology of treatments for viral infection for eliciting the desired activity or biological response in the subject. Dosage regimens can be adjusted to provide an optimum prophylactic or therapeutic response.

An effective amount is also one in which any toxic or detrimental side effects of the compound and/or other biologically active agent is outweighed in clinical terms by therapeutically beneficial effects. A non-limiting range for a therapeutically effective amount of treatments for viral infection within the methods and formulations of the disclosure is about 0.0001 μg/kg body weight to about 10 mg/kg body weight per dose, such as about 0.0001 μg/kg body weight to about 0.001 μg/kg body weight per dose, about 0.001 μg/kg body weight to about 0.01 μg/kg body weight per dose, about 0.01 μg/kg body weight to about 0.1 μg/kg body weight per dose, about 0.1 μg/kg body weight to about 10 μg/kg body weight per dose, about 1 μg/kg body weight to about 100 μg/kg body weight per dose, about 100 μg/kg body weight to about 500 μg/kg body weight per dose, about 500 μg/kg body weight per dose to about 1000 μg/kg body weight per dose, or about 1.0 mg/kg body weight to about 10 mg/kg body weight per dose.

Determination of effective amount is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art, including the EAE model of multiple sclerosis. Using such models, only ordinary calculations and adjustments are required to determine an appropriate concentration and dose to administer a therapeutically effective amount of the treatment.

### EXAMPLES

### Example 1

microRNAs (miRs) are potent regulators of DNA damage responses in the tumor microenvironment. miRs are short 20-22 nucleotide (nt) RNA molecules that regulate gene expression by binding to partially complementary sites in target mRNAs. miRs mediate several physiological processes in endothelial cells. Disclosed herein is data showing that miRs regulate endothelial (EC) DNA damage responses. An expression screen was used to identify miRs induced by radiation, cisplatin or hydrogen peroxide in human ECs. Seven specific miRs unique to intrinsic EC apoptosis pathways regulated by genotoxic stress were identified. In vitro gain-of-function assays show that three of these, miR-21-3p, miR-99b-5p and miR-494-3p lead to endothelial senescence by impairing telomerase function and also inhibit sprouting angiogenesis in vitro. These three miRs each target each member of the MRN (Mre11a-Rad50 and NBS1) complex, a critical part of the cellular DNA repair machinery. The MRN complex plays a vital role in DNA ds break repair, replication, and telomere maintenance. Pulldown of a mutant RNA Induced Silencing Complex (RISC) from cells transfected the miR mimics enriched for the MRN mRNAs suggesting direct miRNA-MRN complex mRNA binding. Consistent with these results, knockdown of the MRN complex recapitulated the effects of the miRs, reproducing the senescence phenotype, angiogenesis inhibition and also impaired telomerase activity.

Since MRE-11a is upregulated in human breast cancer patients, the expression of miR-494-3p in either the tumor ECs or tumor cells was assessed. ISH of a breast cancer tissue array revealed a significant reduction in tumor miR-494-3p levels compared with the adjacent normal tissue. Furthermore, ectopic expression of miR-494-3p diminished breast cancer cell proliferation in 2D and 3D assays. Furthermore, miR-494-3p decreased the growth of murine 4T1 tumors implanted orthotopically in Balb/C mice.

miR-494-3p behaves as a tumor suppressor microRNA by targeting the MRN complex. It thereby induces senescence and cell cycle arrest, inhibits angiogenesis and decreases tumor burden. Restoration of these miRs targeting the MRN complex should be a valuable therapeutic approach likely to synergize with DNA damaging agents in cancer.

### Example 2—microRNAs Inhibit Sprouting Angiogenesis by Targeting the MRN Complex

The MRN complex plays a vital role in DNA double strand break repair and replication. However its role in pathological angiogenesis has not been studied in depth. Disclosed herein is a cohort of microRNAs upregulated under genotoxic stress conditions that target the MRN complex, thereby inducing a senescent phenotype in endothelial cells. Impaired telomerase activity, which is also dependent of MRN complex downregulation was observed. Ectopic overexpression of the disclosed microRNAs inhibits sprouting angiogenesis in vitro as observed in a HUVEC 3D-fibrinogen angiogenesis assay. This angiogenesis inhibition was corroborated using an in vivo model involving Matrigel® plugs. CD31 expression was 50% lower in in plugs treated with one or more of the disclosed microRNAs relative to controls. The results indicate that the disclosed microRNAs have a redundant role in DNA damage and senescence in late damage responses and preventing DNA repair. Use of these microRNAs in pharmaceutical compositions represents an alternative approach to inhibit angiogenesis.

### Example 3—Genotoxic Stress in Endothelial Cells Induce a Cohort of Pro Senescence microRNAs

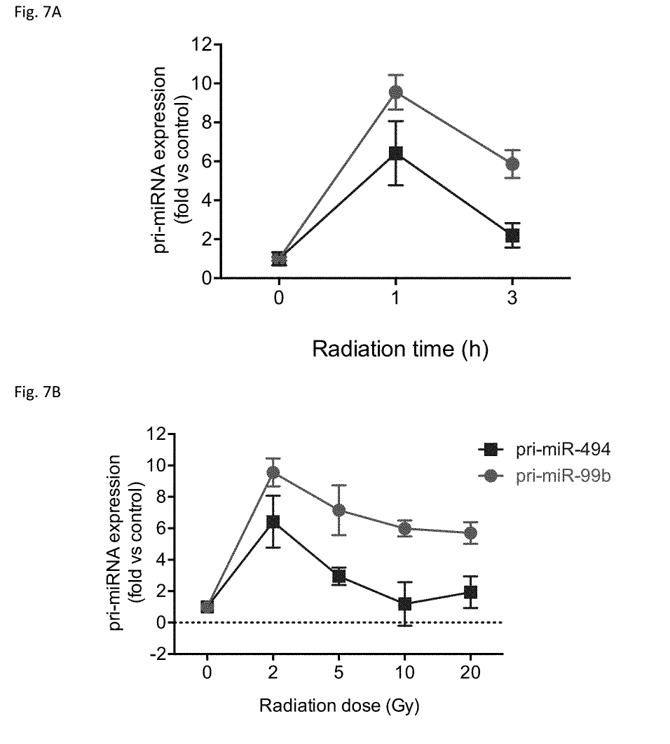

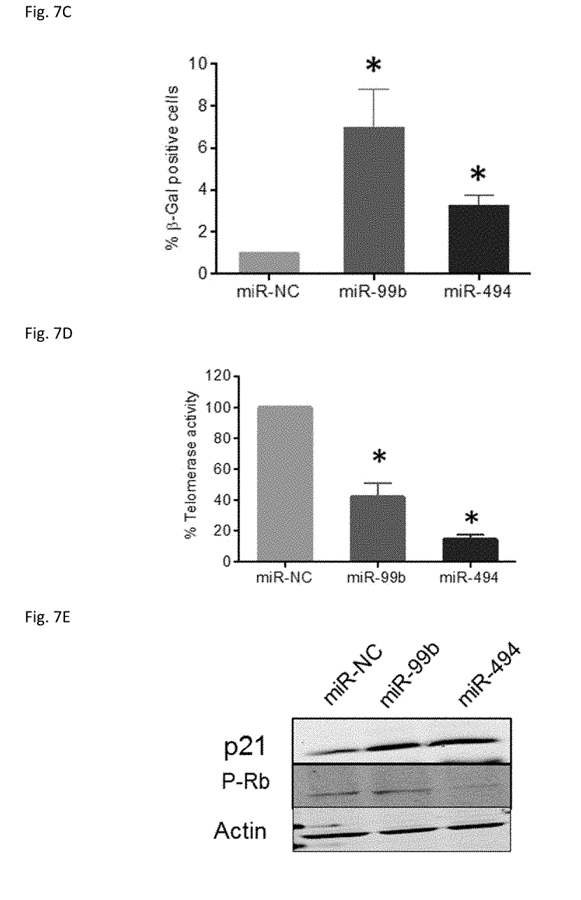

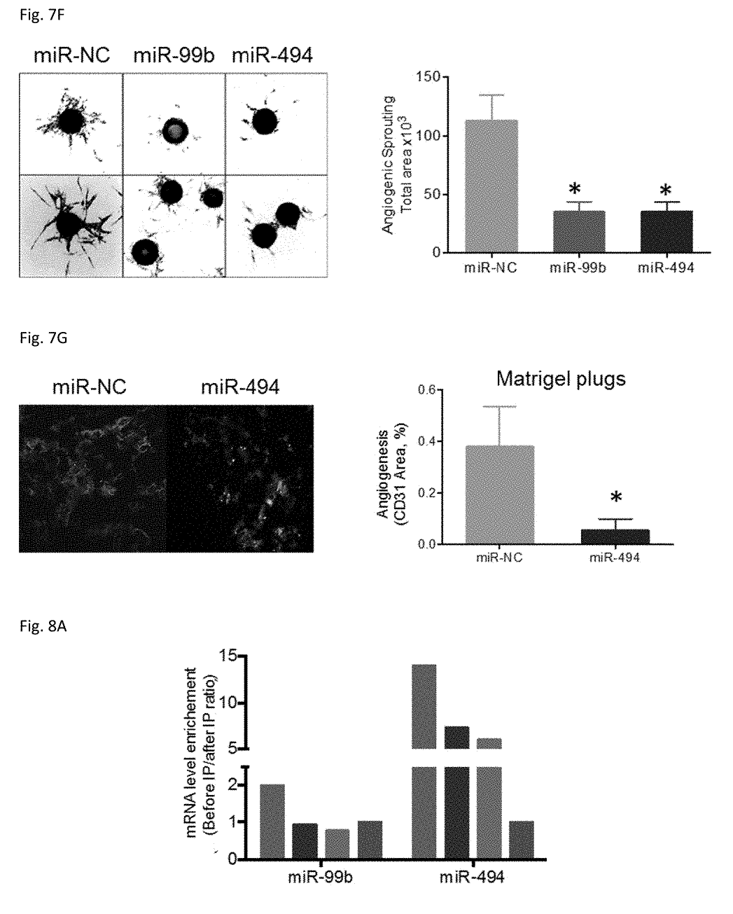

It is well known that endothelial cells respond to stress insults via multiple mechanisms. One such stress insult is radiation. The pre- and the mature form of microRNAs miR-99b and miR-494 are expressed after exposure to a 2 Gy dose of radiation. No additional response was seen with additional radiation. (FIGS. 7A and 7B).

miR-99b and miR 494 were overexpressed in HUVEC cells for 48 hours. The effects of this overexpression on proliferation, apoptosis, and senescence were then assessed. Overexpression of the miRNAs had no effect on apoptosis, but resulted in less proliferation relative to controls. (FIG. 12) This correlates with a higher number of β-galactosidase positive cells in the cells overexpressing the miRNAs relative to controls which in turn suggests a senescence phenotype (FIG. 7C). Impaired telomerase activity was observed after 48 hours of ectopic expression of miR-99b and miR-494. This cell cycle arrest correlates with an greater expression of the cell cycle regulator p21 in cells expressing the miRNAs indicating that the cell cycle arrest is results from an inhibition of P-Rb hyperphosphorylation (FIG. 7D).

### Example 4—Senescence microRNAs are Able to Inhibit Angiogenesis in 3D Co-Culture and in Matrigel Plugs In Vivo

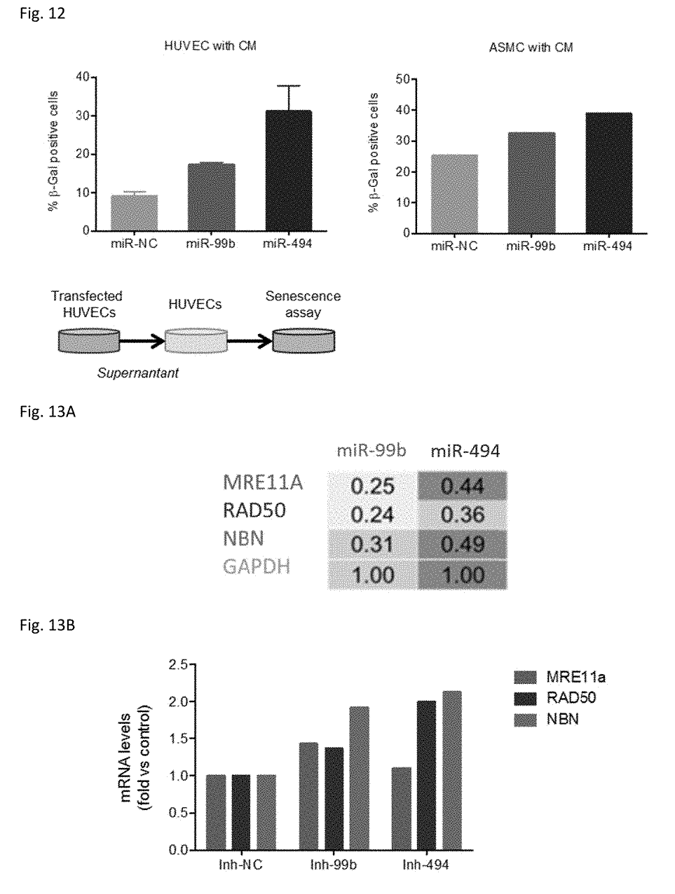

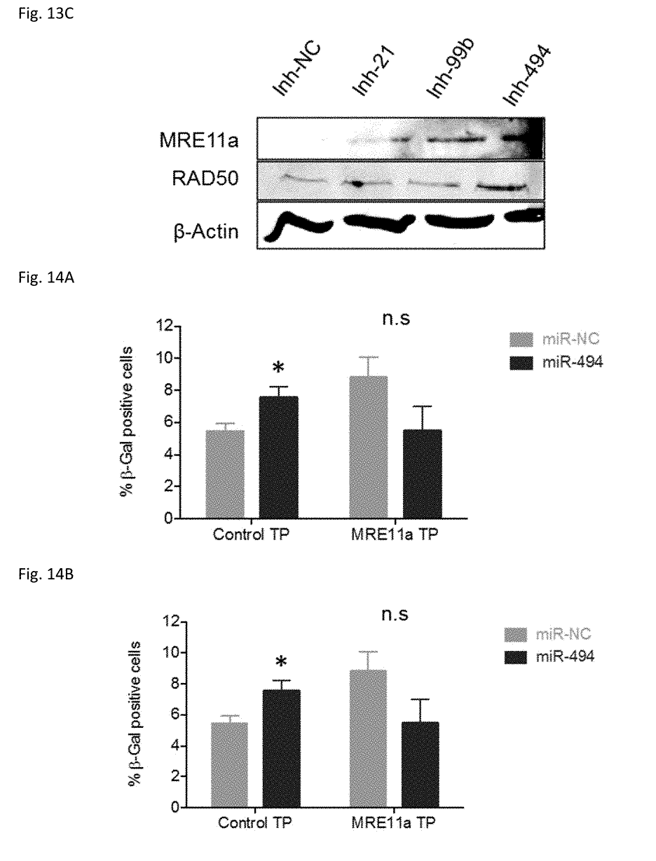

Overexpression of miR-99b and miR-494 inhibited sprouting angiogenesis in a 3D-fibrinogen assay in co-culture with human aortic smooth muscle cells (FIG. 7F). To determine the impact of this phenotype in vivo, a Matrigel plug angiogenesis assay was used. Mice were treated with the endothelial targeted nanoparticle 7C1 to deliver miR-494. 50% less CD31 staining was seen in miR-494 treated plugs relative to controls. In addition, Pecam1 mRNA expression had lower expression in plugs treated with miR-494 relative to controls (FIGS. 13A, 13B, and 13C).

### Example 5—miR-99b and miR-494 Target Essential DNA Repair Complex in Endothelial Cells

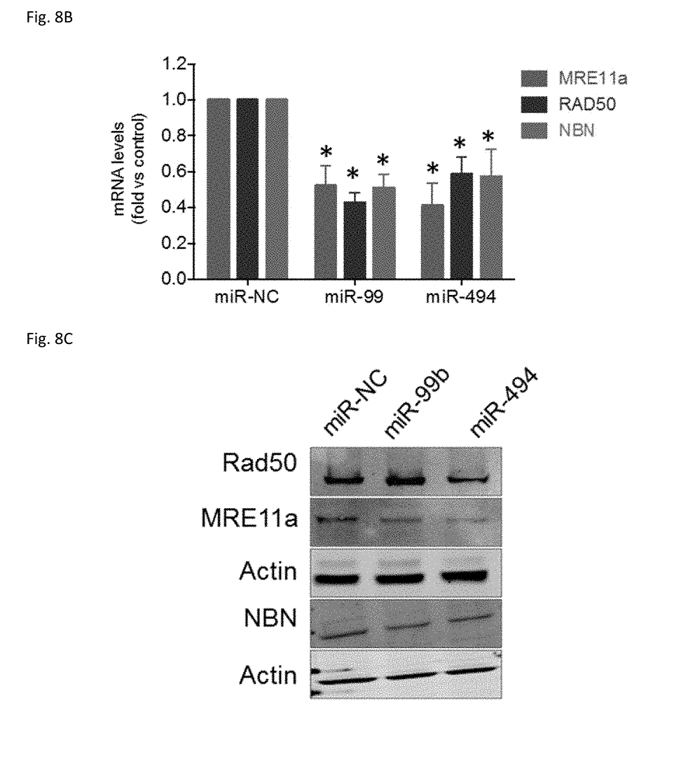

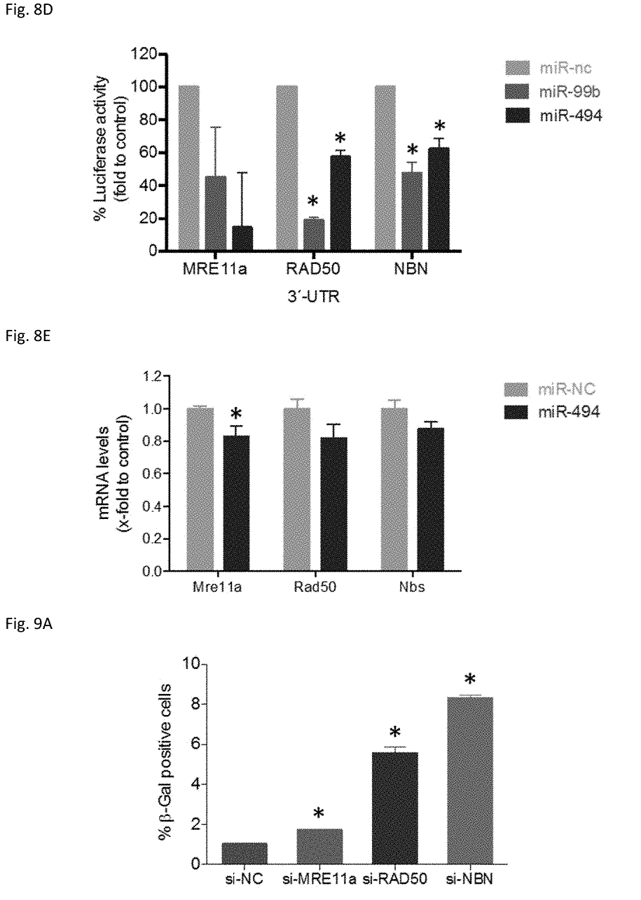

To determine targets of miR-99b and miR-494, the RNAs were ectopically expressed in HUVECs. Targets were analyzed using a DNA damage array 24 hours after transfection. Three common targets for miR-99b and miR-494 were identified: MRE11a, RAD50 and NBN. These three proteins form the MRN complex which is involved in homologous repair and non-homologous end joining. mRNA targets of miR-494 and miR-99b were also assessed using a miR-TRAP assay. Expression of miR-494 resulted in the enrichment of all of MRE11a, RAD50, and NBN relative to controls. For miR-99b, only MRE11a was enriched (FIG. 8A). Blocking of protein expression of these target mRNAs by miR-494 and miR-99b were validated by Western blot (FIG. 8C), by qPCR (FIG. 8B) and immunofluorescence. Finally, the 3′-UTRs of MRE11a, RAD50, and NBN were cloned into luciferase reporter vectors. The luciferase vectors were contrasfected with miR-494 and miR99b into HEK-293T cells for 24 hours. Significantly less luciferase expression was seen in the presence of the microRNAs as indicated (FIG. 8D).

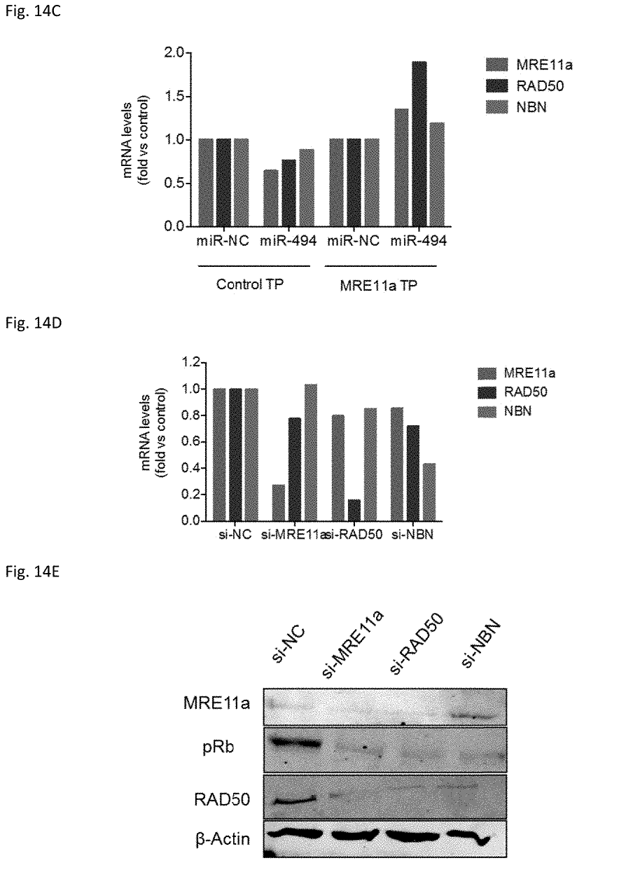

A specific target protector for the miR-494 binding site in MRE11a-3′-UTR was used to further validate the results. Both senescence and telomerase activity caused by expression of miRNA-494 and miRNA 99b were inhibited by the target protector (FIGS. 14A and 14B). mRNA levels of the MRN complex in the presence of MRE11a target protector also showed inhibition of the effects of miR-494. MRN mRNA levels in the RNA isolated from the Matrigel plugs described above were also assessed and significantly less Mre11a expression was observed in the plugs treated with miR-494 (FIG. 8E and S5B).

### Example 6—Disruption of MRN Complex In Vitro Inhibits Sprouting Angiogenesis Through Senescence

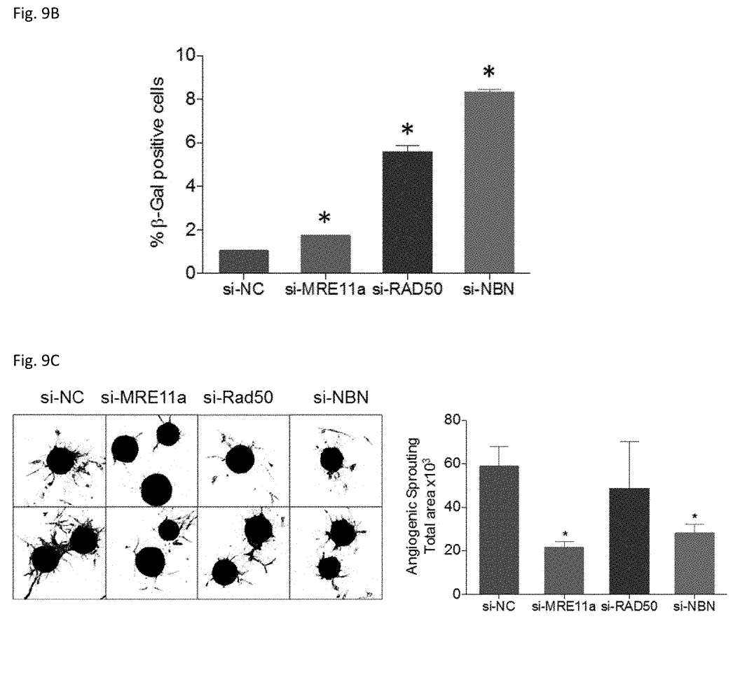

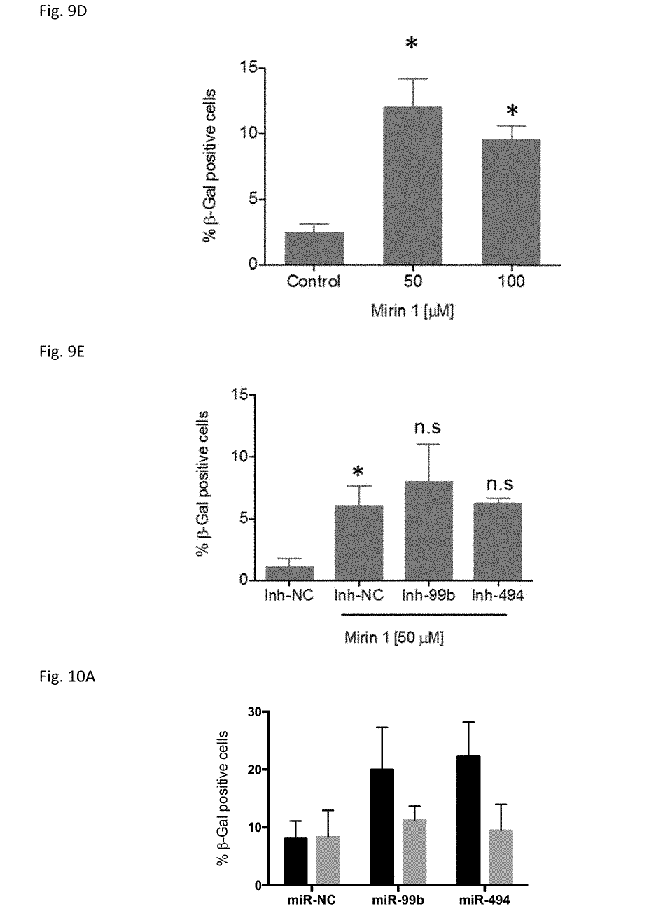

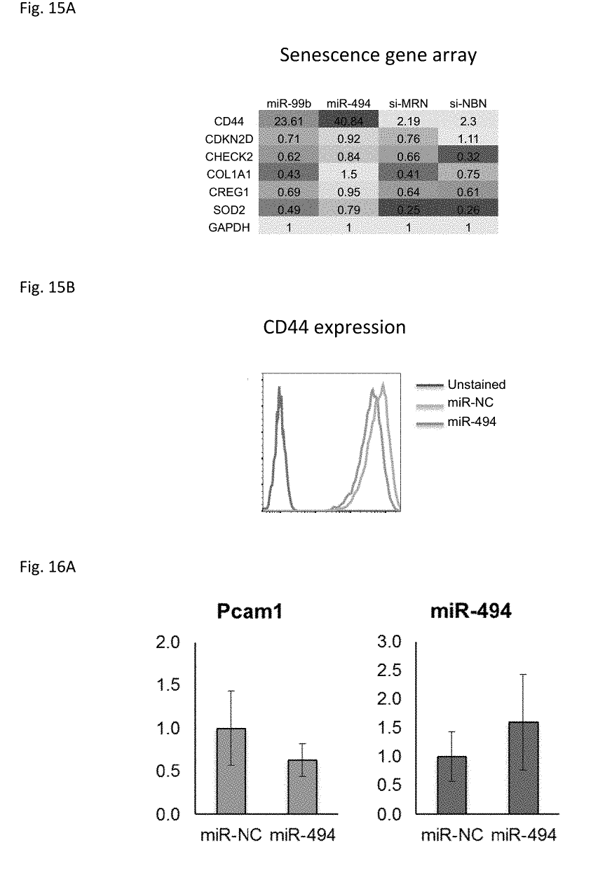

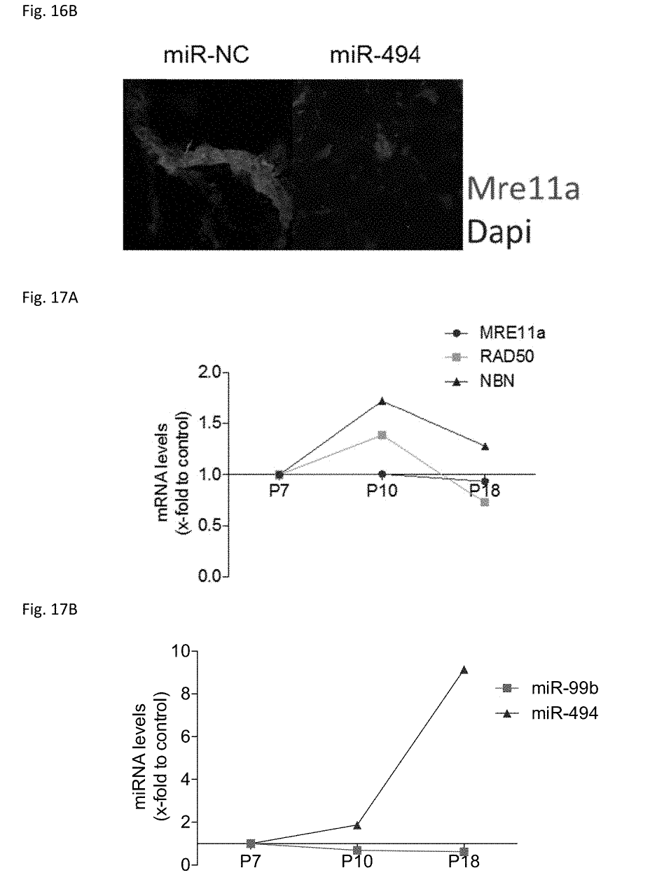

Silencing the expression of all components of the MRN complex results in senescence and impaired telomerase activity (FIGS. 9A, 9B, 14D, and 14E). A small molecule inhibitor specific for MRE11a, called Mirin-1, was also used to validate the MRN-induced senescence phenotype. Mirin-1 (50 uM) in able to induce senescence after 24 hours and that the inhibition of miR-99b or miR-494 is not able to reverse this phenotype. The senescence phenotype was also validated using a Human Senescence gene array. We observed that miR-99b, miR-494, an MRE11a siRNA, and a RAD50 siRNA induced expression of CD44, a marker of senescence. Similar results were also observed in HUVEC that were transfected with CD44 and analyzed by flow cytometry (FIG. 15). Silencing MRN in vitro results in inhibition of sprouting angiogenesis as shown with MRE11a and NBN siRNA transfection (FIG. 9C).

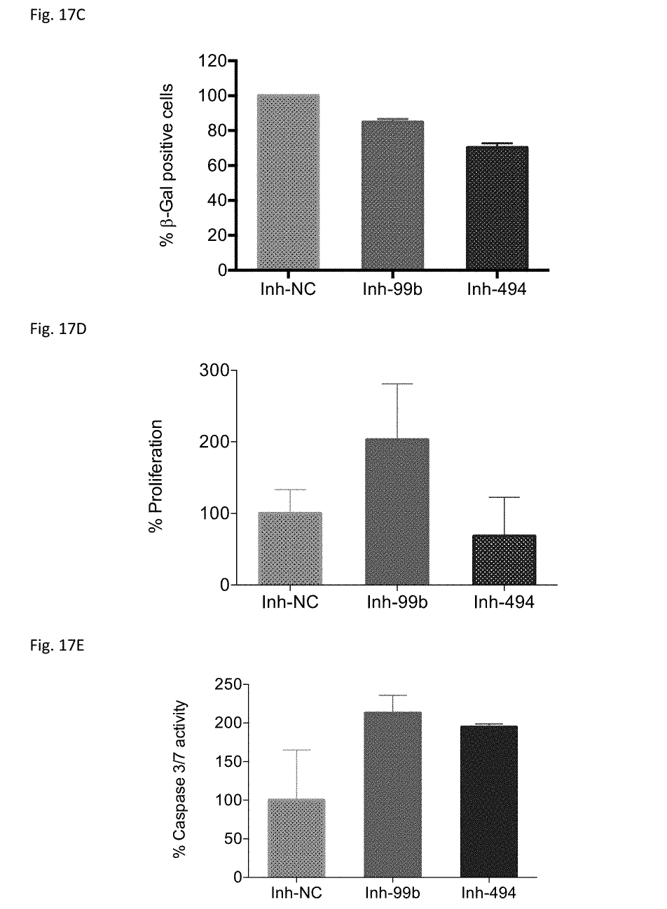

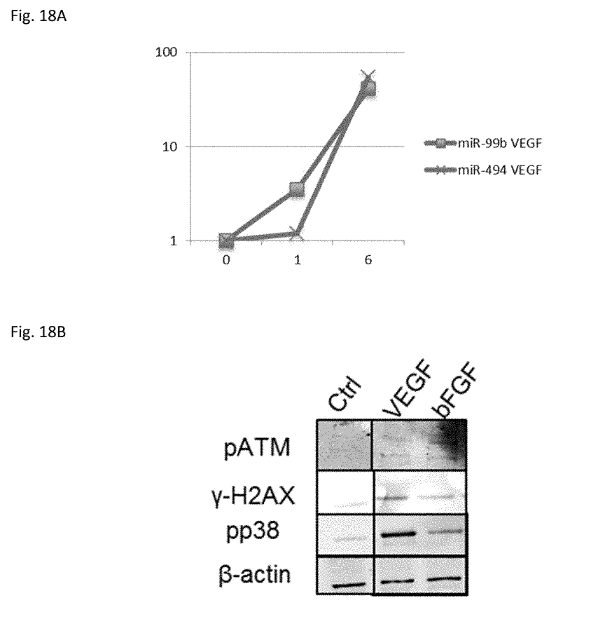

HUVEC in passage 8 to 20, were assessed for expression of miR-99b and miR-494. Expression of miR-99b and miR-494 were observed to increase with an higher number of passages while expression of the genes of the MRN complex were observed to decrease with a higher number of passages (FIGS. 17A and 17B). A β-galactosidase assay confirmed that miR-494 and miR-99b restored a senescence phenotype in old HUVECs, without dramatically changing proliferation, suggesting a more pro-apoptotic pathway (FIGS. 17C and 17D).

### Example 6—MRN-VEGF Codependency

Recent publications have shown than disruption of DNA damage response (DDR) modulates pathological angiogenesis modulating VEGF pathway. It is disclosed herein that short-pulse VEGF in HUVEC induces induce phosphorylation of ATM and y-H2AX, two downstream effectors of the MRN complex. As a result, miR-99b and miR-494 expression were assessed 3 and 6 hours after treatment with VEGF (50 ug/ml) in starved HUVECs, resulting in a time dependent increase in expression.

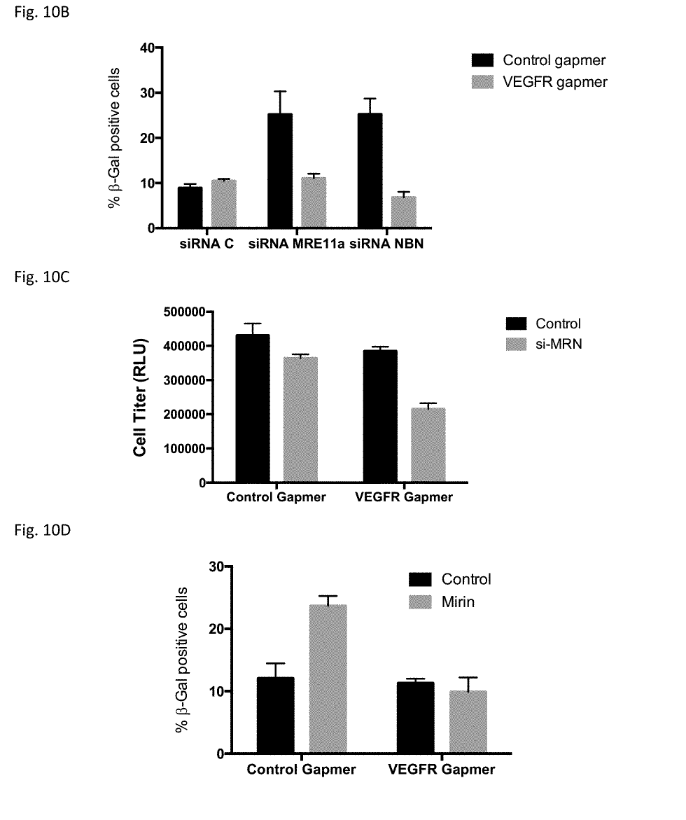

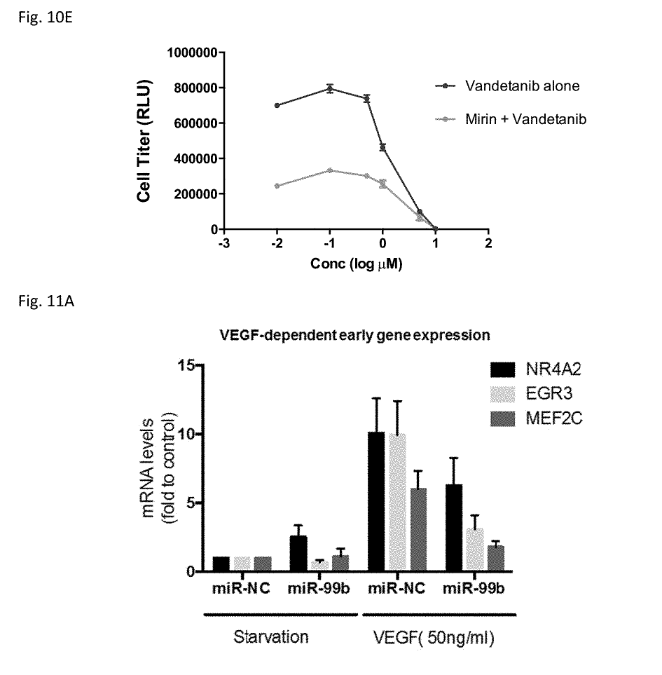

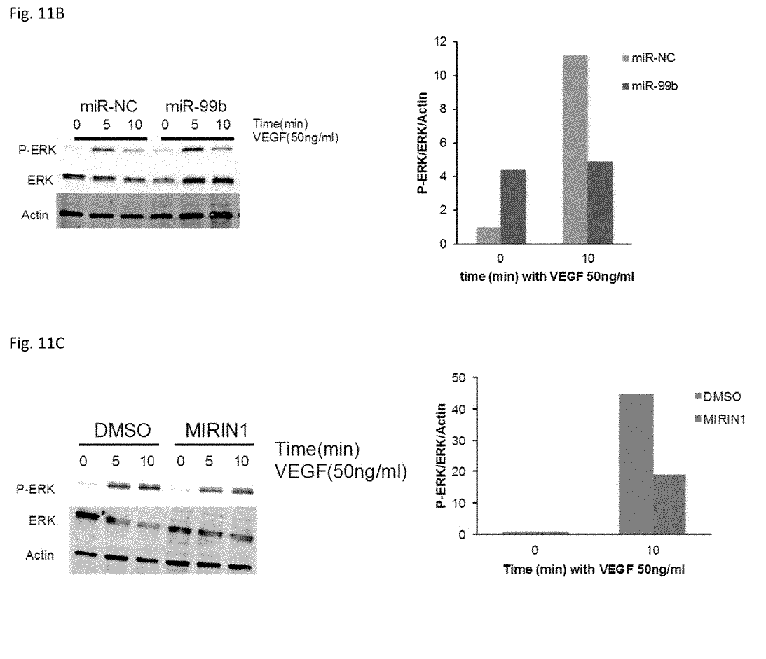

VEGF-receptor 2 (VEGFR2) was cotransfected with miR-99b, miR-494, or siRNAs that silence expression of the MRN complex into HUVECs. This showed that the VEGF-VEGFR active pathway is necessary for senescence after MRN disruption (FIGS. 10A and 10B). Similar results were seen with the specific small molecule inhibitor Mirin-1 (FIG. 10C). Less proliferation was observed with VEGFR2 and MRN specific silencing relative to controls (FIG. 10C) and also with Mirin-1 and Vandetanib, a small molecule inhibitor for VEGFR2 (FIG. 10E).

Finally, miR-99b expression was shown to block a specific-VEGF-early gene signature. Accordingly, cells expressing miR-99b also displayed a lower rate of phosphorylation of ERK after 10 min of VEGF treatment and treatment with Mirin-1 showed decreased ERK phosphorylation in HUVECs treated with VEGF.

### Example 7—Cell Culture and Reagents

HUVECs (Lonza) were cultured in EBM-2 media (Lonza) supplemented with bullet kit and 10% Fetal Calf Serum (Hyclone). ASMA (Lonza) were cultured in Medium 231, supplemented with Smooth muscle growth serum (GIBCO). Cells were tested and found negative for mycoplasma contamination before use in the assays described. Mirin-1 and Vandetanib were purchased from Cayman Chem and Selleckchem respectively. VEGF was purchased from PeproTech, Inc.

### Example 8—miRs/Anti-miRs/siRNAs

miR-494 and miR-99b mimics, inhibitors and respective controls were purchased from Life Technologies and Exiqon. For in vivo studies, high-performance liquid chromatography-purified miRs were purchased from Life Technologies. siRNAs against MRE11a, RAD50 and NBN were purchased from Life Technologies. Gapmer MRE11a was purchased from Exiqon.

### Example 9—Vectors/Plasmids

MRE11a Luciferase-3-UTR plasmid was purchased from SwitchGear Genomics. RAD50 and NBN luciferase constructs were purchased as pmiR-REPORT vector (Ambion). Luciferase assay reagents were purchased from Switch Gear Genomics and Promega.

### Example 10—Transfection of miRNA Mimics and miRNA Inhibitors

Cells were transfected at 50-60% confluence using standard forward transfection protocols using RNAimax reagent (Life Technologies) for miRs/siRNAs and Lipofectamine 2000 for plasmid or plasmid RNA dual transfections. Typically 50 nM RNA and 1-2 mg plasmid DNA were used for transfections. Target protectors were transfected at a concentration of 50 nM or equivalent to the miR amounts.

### Example 11—Radiation of Cells

Cells were irradiated using a Shepherd 137cesium irradiator at a rate of B166 cGy min 1.

### Example 12—β-Gal Senescence Assay

HUVEC were transfected with the correspondent microRNA or siRNA during 48 hours. After this time cells were washed whit cold PBS and then stain for β-galactosidase activity following manufacture protocol (Senescence Cells Histochemical Staining Kit, Sigma).

### Example 13—Telomerase Activity Assay

Cells were transfected with microRNAs and/or siRNAs as indicated for 24 hours. Cells were lysed and processed according to the manufacturer's instructions (Quantitative Telomerase Detection Kit, Allied Biotech). The telomerase activity in the cell extract was determined through its ability to synthesize telomeric repeats onto an oligonucleotide substrate. The resulting products were subsequently amplified using the polymerase chain reaction (PCR).

### Example 14—Western Blot and Densitometric Analysis

After treatment, cells were washed in phosphate-buffered saline (PBS) and prepared in RIPA lysis buffer (Sigma) supplemented with Complete Protease inhibitor cocktail (ROCHE) and Phosphatase inhibitors cocktail 2 and 3 (Sigma). Cells were harvested by scraping, and proteins were analyzed by Western blot. Equivalent amounts of protein were loaded on a 4-12% gradient SDS-polyacrylamide gel (BioRAD) and transferred for 30 min to a TransBlot turbo (BioRAD) nitrocellulose membrane. Membranes were blocked in 5% milk or 3% BSA and incubated with antibodies as indicated: Mre11a (Cell Signaling, 4847, 1:1000), RAD50 (Cell Signaling, 3427, 1:1000), NBS1 (Cell Signaling, 14956, 1:500), p-ATM (Cell Signaling, 4526, 1:500), H2AX (Abcam 11174, 1:500), p21 (Cell Signaling, 2947, 1:1000), P-Rb (Cell Signaling, 9301, 1:500), ERK1/2 (Cell Signaling, 9102, 1:1000), P-ERK1/2 (9101, 1:1000). β-actin (Sigma, A5316, 1:10,000 1 h RT) was used as housekeeping controls for the total levels of protein loaded. Membranes were washed in TBST and incubated with secondary antibodies from Licor Biosciences were used goat anti mouse 925-68020 (1:15,000) and goat anti rabbit 925-32211 (1:15,000). Blots were scanned on the Licor Odyssey scanner according to manufacturer's instructions.

### Example 15—RNA Extraction, RT-PCR, miR Profiling

Total RNA and microRNA were isolated using miRVana microRNA isolation kit (Ambion). Reverse transcription was performed using TaqMan™ Advanced cDNA Synthesis Kit (Life Tech) according to the manufacture instructions. RT-PCR was performed using multiplexed TaqMan primers (Applied Biosystems). The relative quantification of gene expression was determined using the 2-DDCtmethod. Using this method, we obtained the fold changes in gene expression normalized to an internal control gene, GAPDH or U6 snRNA, respectively. A DNA damage array (LifeTech) was used for targeting analysis and a Human Cellular Senescence array (SA Biosciences) was used for senescence phenotype profiling.

### Example 16—miR-TRAP/RISC TRAP Assay

293 T cells were co-transfected with a plasmid coding for a flag-tagged dominant negative GW418 mutant (Clontech) along with a control mimic, miR-99b or miR-494 mimic according to kit instructions. Twenty-four hours later the RNA protein complexes were crosslinked and the RISC complex was immunoprecipitated using an anti-FLAG antibody and RNA was isolated for quantitative real-time PCR of target genes. The fold enrichment was calculated using pre and post IP controls as well as normalization to the control mimic pull-downs.

### Example 17—3-D Angiogenic Sprouting Assay

Early passage HUVECs were coated on cytodex-3 (GE Healthcare) beads at a density of 10 million cells/40 μl beads and incubated in suspension for 3-4 hours with gentle mixing every hour. They were plated on TC treated 6 well dishes overnight and resuspended in a 2 mg/ml fibrin gel with 200,000 human smooth muscle cells. The gel was allowed to polymerize and complete EGM-2 media was added. Sprouts were visualized from days 3-4 via confocal imaging after overnight incubation with FITC labeled Ulex europaeus lectin (Vector labs). Immunofluorescence imaging was performed on a Yokogawa CSU-W1 spinning disk confocal microscope with 20 0.45 Plan Fluor objective (Nikon).

### Example 18—Flow Cytometry

CD44 expression was analyzed by flow cytometry in HUVECs transfected with miR-494 for 48 hours. Cells were washed in PBS and tripsinazed. Cells were incubated in blocking solution (0.5% BSA/10% goat serum) for 30 min and 1 hour in primary antibody-PEcy7 conjugated (CD44, BD, 560533) prepared in blocking solution. Then cells were washed in PBS 3×. Cells were analyzed in a CANTOII equipment.

### Example 19—Immunofluorescence and Microscopy

In some experiments, CD31a and Mre11a were visualized using immunofluorescence staining from OCT sections of tumor tissue. Slides were fixed with 4% PFA and stained overnight for CD31, Mre11a, or ASMC. Imaging was performed on a Nikon Spectral C1 confocal microscope (Nikon C1si with EZC1 acquisition software, Nikon Instruments) with Plan Apo 10×/0.45 air, Plan Apo 20×/0.75 air, and Plan Apo 60×/1.40 oil objective lenses (Nikon). Some immunofluorescence imaging was performed on a Yokogawa CSU-W1 spinning disk confocal microscope with 20 0.45 Plan Fluor objective (Nikon). All images were taken with a channel series. Images were analysed with Image J software for quantitation.

### Example 20—In Vivo Assays

Immune-compromised 8-10 week old female nu/nu mice purchased from Jackson Labs. Growth factor reduced Matrigel (BD) with 400 ng ml 1 recombinant human bFGF (Millipore) was injected subcutaneously in nu/nu mice. Mice were injected i.v with 7C1-nanoparticles containing miR-494 or control miR (0.7 mg/kg, i.v) 3 or 4 days after plugs were implanted. At day 7 mice were harvest and lysed to obtained RNA or frozen in OCT for tissue staining.

### Example 21—Statistics

All statistical analysis was performed using Excel (Microsoft) or Prism (GraphPad). Sample size was estimated using a DSS research tool to detect effect size of 25% with an a-error of 5% and b-error of 10%. Two-tailed Student's t-test or Mann-Whitney U-test was used to calculate statistical significance. Data that was not normally distributed as assessed by Shapiro-wilk test (Excel, Real statistics add-in) was evaluated using U-test. Variance was similar between treatment groups.

1. A pharmaceutical composition comprising an effective amount of a first polyribonucleotide with a sequence of SEQ ID NO: 1, SEQ ID NO: 2, and/or SEQ ID NO: 3 and a pharmaceutically acceptable carrier provided that the polyribonucleotide is no more than 35 nucleotides in length.

2. The pharmaceutical composition of claim 1 where the one or more of SEQ ID NO: 1, SEQ ID NO: 2, and/or SEQ ID NO: 3 are provided in a recombinant expression vector.

3. The pharmaceutical composition of claim 2 comprising a plasmid or viral vector.

4. The pharmaceutical composition of claim 3 comprising an adenoviral, adeno associated viral, lentiviral, herpesviral, or poxviral vector.

5. The pharmaceutical composition of claim 1 where the first polyribonucleotide comprises a modified nucleotide.

6. The pharmaceutical composition of claim 1 where the first polyribonucleotide has a sequence of SEQ ID NO: 1, further comprising a second oligonucleotide with a sequence of SEQ ID NO: 2 or SEQ ID NO: 3.

7. A method of treating a cancer characterized by aberrant activity of an MRN complex in a subject, the method comprising: administering the pharmaceutical composition of claim 1 to the subject.

8. A method of inhibiting angiogenesis in a subject, the method comprising administering the pharmaceutical composition of claim 1 to the subject.


SEQUENCE LISTING (3 sequences)

SEQ ID NO: 1 - RNA, 22 nt, Homo sapiens
ugaaacauacacgggaaaccuc

SEQ ID NO: 2 - RNA, 21 nt, Homo sapiens
caacaccagucgaugggcugu

SEQ ID NO: 3 - RNA, 22 nt, Homo sapiens
cacccguagaaccgaccuugcg